(19) United States
(12) Patent Application Publication
(10) Pub. No.: US 2026/0021167 A1
(43) Pub. Date: Jan. 22, 2026

(54) COMPOSITIONS AND METHODS FOR TREATING OR PREVENTING NEURODEGENERATIVE DISEASES

(71) Applicant: Delaware State University, DOVER, DE (US)

(72) Inventor: YongHwan Kim, Boise, ID (US)

(73) Assignee: Delaware State University, Dover, DE (US)

(21) Appl. No.: 18/875,489

(22) PCT Filed: Jun. 16, 2023
(86) PCT No.: PCT/US2023/025507

§ 371 Date: Dec. 16, 2024

Related U.S. Application Data
(60) Provisional application No. 63/352,866, filed on Jun. 16, 2022.

(51) Int. Cl. A61K 38/47; A61P 25/28; C12N 9/24

(52) U.S. Cl. CPC A61K 38/47; A61P 25/28; C12N 9/2497; C12Y 302/02022

(57) ABSTRACT

The present invention provides methods for treating a neurodegenerative disease in a subject, preventing a neurodegenerative disease in a subject, increasing neurons in a region of the brain of a subject, increasing small ubiquitin-like modifier 1 (SUMO-1) in a region of the brain of a subject, reducing Sentrin-specific protease 1 (SENP-1) in a region of the brain of a subject, reducing Serine 129 phosphorylated alpha-synuclein in a region of the brain of a subject, reducing SENP-1 nuclear translocalization from the cytosol and/or reducing protein aggregates in a region of the brain of a subject. The method comprises administering to the subject an effective amount of a pharmaceutical composition comprising a specific SENP-1 inhibitor. The specific SENP-1 inhibitor may be selected from the group consisting of SUMO-2 aldehyde, momordin lc, streptonigrin, hinokiflavone, siRNA of SENP-1, and a combination thereof. Also provided is the pharmaceutical composition.

### CROSS-REFERENCE TO RELATED APPLICATION

This application claims priority to U.S. Provisional Application No. 63/352,866 filed Jun. 16, 2022, the contents of which are incorporated herein by reference in their entireties for all purposes.

### STATEMENT REGARDING FEDERALLY SPONSORED RESEARCH

This invention was made with government support under Grant Nos. R15NS121784 and 5P20GM103653 awarded by National Institute of Health (NIH) and National Institute of Health, Centers of Biomedical Research Excellence (NIH-COBRE), respectively. The government has certain rights in the invention.

### FIELD OF THE INVENTION

This invention relates to compositions and methods for treating or preventing neurodegenerative diseases, for example, Parkinson's disease (PD).

### BACKGROUND OF THE INVENTION

Parkinson's disease (PD) is the most prevalent motor neurodegenerative disease, which is a chronic and progressive degenerative disease of the brain that impairs motor control, speech, and other functions. In particular, it is characterized by (1) slowing down of all movements (bradykinesia), quiet and monotonous speech (akinesia or hypokinesia), absence of the physiological associated movements, a stooped posture, a small-step, partially shuffling gait, handwriting which becomes smaller as the writing continues, uncontrollable disturbances in movement, with a tendency to fall forward to the side or backward, (2) rigidity of the musculature (rigor), and (3) coarse resting tremor (trembling). Parkinson's disease is a disease that occurs relatively frequently and develops in approx. 1% of individuals aged over 60 with higher prevalence in men than women. The disease is caused by loss of dopamine in the striatum, resulting in neuronal loss in the Substantia Nigra pars compacta (SNpc). The primary reason for loss of dopamine remains to be somewhat elusive, although 10-15% of cases are known to be genetic. Then, the majority cases are sporadic without knowing a cause.

The motor symptoms of PD are caused by loss of nerve cells that secrete dopamine in a tiny midbrain area called the SNpc. For reasons that are not fully understood, these nerve cells are especially vulnerable to damages of various sources including drugs, head trauma, pollutants, heavy metal exposure and other environmental factors. The term Parkinsonism is used for any process that destroys large numbers of these neurons and thereby causes the same characteristic symptoms. Parkinson's disease, or more fully, idiopathic Parkinson's disease, is typically diagnosed when no specific physical cause for the loss of dopamine neurons can be identified.

The term Parkinsonism is used for symptoms of tremor, stiffness, and slowing of movement caused by loss of dopaminergic neurons in the substantia nigra. “Parkinson's disease” is the synonym of “primary Parkinsonism”, i.e. isolated Parkinsonism due to a neurodegenerative process without any secondary systemic cause. In some cases, it would be inaccurate to say that the cause is “unknown” because 10-15% of cases are caused by identifiable genetic mutations. It is possible for a patient to be initially diagnosed with Parkinson's disease but then to develop additional features requiring revision of the diagnosis.

There are other disorders called Parkinson-plus diseases. These include: multiple system atrophy (MSA), progressive supranuclear palsy (PSP), corticobasal degeneration (CBD), and dementia with Lewy bodies (DLB). Lewy bodies are abnormal aggregates of protein that develop inside nerve cells. Most idiopathic Parkinson's disease patients also have Lewy bodies in their brain tissue, but the distribution is denser and more widespread in DLB. Even so, the relationship between Parkinson's disease, Parkinson's disease with dementia, and dementia with Lewy bodies (DLB) might be most accurately conceptualized as a spectrum, with a discrete area of overlap between each of those three disorders.

The Parkinson-plus diseases may progress more quickly than typical idiopathic Parkinson disease. If cognitive dysfunction occurs before or very early in the course of the movement disorder, then DLB may be suspected. Early postural instability with minimal tremor especially in the context of ophthalmoparesis should suggest PSP. Early autonomic dysfunction including erectile dysfunction and syncope may suggest MSA. The presence of extreme asymmetry with patchy cortical cognitive defects such as dysphasia and apraxia especially with “alien limb” phenomena should suggest CBD.

There remains a need for methods for treating or preventing Parkinson's disease (PD) or other neurodegenerative diseases by targeting a biological molecule associated with the pathological conditions of the disease.

### SUMMARY OF THE INVENTION

The present invention relates to pharmaceutical compositions and methods for treating or preventing a neurodegenerative disease in a subject in need thereof.

A pharmaceutical composition is provided. The pharmaceutical composition comprises a specific Sentrin-specific protease 1 (SENP-1) inhibitor in an amount effective for (a) treating a neurodegenerative disease in a subject; (b) preventing a neurodegenerative disease in a subject; (c) increasing neurons in a region of the brain of a subject; (d) increasing small ubiquitin-like modifier 1 (SUMO-1) in a region of the brain of a subject; (e) reducing Sentrin-specific protease 1 (SENP-1) in a region of the brain of a subject; (f) reducing Serine 129 phosphorylated alpha-synuclein in a region of the brain of a subject; and/or (g) reducing protein aggregates in a region of the brain of a subject. The region of the brain may comprise midbrain, striatum (STR), cortex or a combination thereof. The subject may be a human. The subject may have suffered from a neurodegenerative disease. The subject may be predisposed to a neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body, and Parkinson-plus disease. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. The composition may comprise the specific SENP-1 inhibitor in an amount ranging from 1 mg to 100 g.

A method is provided for treating a neurodegenerative disease in a subject in need thereof. The treatment method comprises administering to the subject an effective amount of a pharmaceutical composition comprising a specific Sentrin-specific protease 1 (SENP-1) inhibitor. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. The composition may comprise the specific SENP-1 inhibitor in an amount ranging from 1 mg to 100 g. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body, and Parkinson-plus disease. The subject may be a human. The subject may have suffered from the neurodegenerative disease.

The treatment method may further comprise mitigating severity of a symptom associated with the neurodegenerative disease.

The treatment method may further comprise reversing the severity of the symptom.

The treatment method may further comprise improving mobility of the subject.

The treatment method may further comprise (a) increasing neurons in a region of the brain of the subject; (b) increasing small ubiquitin-like modifier 1 (SUMO-1) in a region of the brain of the subject; (c) reducing Sentrin-specific protease 1 (SENP-1) in a region of the brain of the subject; (d) reducing Serine 129 phosphorylated alpha-synuclein in a region of the brain of the subject; and/or (e) reducing protein aggregates in a region of the brain of the subject. The region of the brain may comprise midbrain, striatum (STR), cortex, or a combination thereof.

The treatment method may further comprise blocking mRNA expression of the SENP-1. The treatment method may further comprise administering the pharmaceutical composition to the subject orally. The treatment method may further comprise administering the pharmaceutical composition to the subject at a dose of the specific SENP-1 inhibitor at 1-500 mg per kg of the subject's body weight. The treatment method may further comprise administering the pharmaceutical composition to the subject at a dose of the specific SENP-1 inhibitor at 1-500 mg per kg of the subject's body weight daily for 1-10 weeks.

A method is provided for preventing a neurodegenerative disease in a subject in need thereof. The prevention method comprises administering to the subject an effective amount of a pharmaceutical composition comprising a specific Sentrin-specific protease 1 (SENP-1) inhibitor. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. The composition may comprise the specific SENP-1 inhibitor in an amount ranging from 1 mg to 100 g. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body, and Parkinson-plus disease. The subject may be a human. The subject may be predisposed to the neurodegenerative disease.

The prevention method may further comprise delaying onset of a symptom associated with the neurodegenerative disease in the subject.

The prevention method may further comprise delaying onset of motor dysfunction in the subject.

The prevention method may further comprise delaying onset of (a) loss of neuron in a region of the brain of the subject; (b) decrease of small ubiquitin-like modifier 1 (SUMO-1) in a region of the brain of the subject; (c) increase of Sentrin-specific protease 1 (SENP-1) in a region of the brain of the subject; (d) increase of serine 129 phosphorylated alpha-synuclein in a region of the brain of the subject; and/or (e) increase of protein aggregates in a region of the brain of the subject. The region of the brain may comprise midbrain, striatum (STR), cortex, or a combination thereof.

The prevention method may further comprise blocking mRNA expression of the SENP-1. The prevention method may further comprise administering the pharmaceutical composition to the subject orally. The prevention method may further comprise administering the pharmaceutical composition to the subject at a dose of the specific SENP-1 inhibitor at 1-500 mg per kg of the subject's body weight. The prevention method may further comprise administering the pharmaceutical composition to the subject at a dose of the specific SENP-1 inhibitor at 1-500 mg per kg of the subject's body weight daily for 1-10 weeks.

A method is provided for increasing neurons in a region of the brain of a subject in need thereof. The neuron increase method comprises administering to the subject an effective amount of a pharmaceutical composition comprising a specific Sentrin-specific protease 1 (SENP-1) inhibitor. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. The composition may comprise the specific SENP-1 inhibitor in an amount ranging from 1 mg to 100 g. The region of the brain may comprise midbrain, striatum (STR), cortex or a combination thereof. The region of the brain may comprise striatum (STR), substantia nigra compacta (SNc) in midbrain, or a combination thereof. The region of the brain may comprise midbrain. The region of the brain may comprise cortex. The subject may be a human. The subject may have suffered from a neurodegenerative disease. The subject may be predisposed to a neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body, and Parkinson-plus disease.

The neuron increase method may further comprise blocking mRNA expression of the SENP-1. The neuron increase method may further comprise administering the pharmaceutical composition to the subject orally. The neuron increase method may further comprise administering the pharmaceutical composition to the subject at a dose of the specific SENP-1 inhibitor at 1-500 mg per kg of the subject's body weight. The neuron increase method may further comprise administering the pharmaceutical composition to the subject at a dose of the specific SENP-1 inhibitor at 1-500 mg per kg of the subject's body weight daily for 1-10 weeks.

A method is provided for increasing small ubiquitin-like modifier 1 (SUMO-1) in a region of the brain of a subject in need thereof. The SUMO-1 increase method comprises administering to the subject an effective amount of a pharmaceutical composition comprising a specific Sentrin-specific protease 1 (SENP-1) inhibitor. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. The composition may comprise the specific SENP-1 inhibitor in an amount ranging from 1 mg to 100 g. The region of the brain may comprise midbrain, striatum (STR), cortex or a combination thereof. The region of the brain may comprise striatum (STR), substantia nigra compacta (SNc) in midbrain, or a combination thereof. The region of the brain may comprise midbrain. The region of the brain may comprise cortex. The subject may be a human. The subject may have suffered from a neurodegenerative disease. The subject may be predisposed to a neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body, and Parkinson-plus disease.

The SUMO-1 increase method may further comprise blocking mRNA expression of the SENP-1. The SUMO-1 increase method may further comprise administering the pharmaceutical composition to the subject orally. The SUMO-1 increase method may further comprise administering the pharmaceutical composition to the subject at a dose of the specific SENP-1 inhibitor at 1-500 mg per kg of the subject's body weight. The SUMO-1 increase method may further comprise administering the pharmaceutical composition to the subject at a dose of the specific SENP-1 inhibitor at 1-500 mg per kg of the subject's body weight daily for 1-10 weeks.

A method is provided for reducing Sentrin-specific protease 1 (SENP-1) in a region of the brain of a subject in need thereof. The SENP-1 reduction method comprises administering to the subject an effective amount of a pharmaceutical composition comprising a specific Sentrin-specific protease 1 (SENP-1) inhibitor. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. The composition may comprise the specific SENP-1 inhibitor in an amount ranging from 1 mg to 100 g. The region of the brain may comprise midbrain, striatum (STR), cortex or a combination thereof. The region of the brain may comprise striatum (STR), substantia nigra compacta (SNc) in midbrain, or a combination thereof. The region of the brain may comprise midbrain. The region of the brain may comprise cortex. The subject may be a human. The subject may have suffered from a neurodegenerative disease. The subject may be predisposed to a neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body, and Parkinson-plus disease.

The SENP-1 reduction method may further comprise blocking mRNA expression of the SENP-1. The SENP-1 reduction method may further comprise administering the pharmaceutical composition to the subject orally. The SENP-1 reduction method may further comprise administering the pharmaceutical composition to the subject at a dose of the specific SENP-1 inhibitor at 1-500 mg per kg of the subject's body weight. The SENP-1 reduction method may further comprise administering the pharmaceutical composition to the subject at a dose of the specific SENP-1 inhibitor at 1-500 mg per kg of the subject's body weight daily for 1-10 weeks.

A method is provided for reducing Serine 129 phosphorylated alpha-synuclein in a region of the brain of a subject in need thereof. The Serine 129 phosphorylated alpha-synuclein reduction method comprises administering to the subject an effective amount of a pharmaceutical composition comprising a specific Sentrin-specific protease 1 (SENP-1) inhibitor. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. The composition may comprise the specific SENP-1 inhibitor in an amount ranging from 1 mg to 100 g. The region of the brain may comprise midbrain, striatum (STR), cortex or a combination thereof. The region of the brain may comprise striatum (STR), substantia nigra compacta (SNc) in midbrain, or a combination thereof. The region of the brain may comprise midbrain. The region of the brain may comprise cortex. The subject may be a human. The subject may have suffered from a neurodegenerative disease. The subject may be predisposed to a neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body, and Parkinson-plus disease.

The Serine 129 phosphorylated alpha-synuclein reduction method may further comprise blocking mRNA expression of the SENP-1. The Serine 129 phosphorylated alpha-synuclein reduction method may further comprise administering the pharmaceutical composition to the subject orally. The Serine 129 phosphorylated alpha-synuclein reduction method may further comprise administering the pharmaceutical composition to the subject at a dose of the specific SENP-1 inhibitor at 1-500 mg per kg of the subject's body weight. The Serine 129 phosphorylated alpha-synuclein reduction method may further comprise administering the pharmaceutical composition to the subject at a dose of the specific SENP-1 inhibitor at 1-500 mg per kg of the subject's body weight daily for 1-10 weeks.

A method is provided for reducing protein aggregates in a region of the brain of a subject in need thereof. The protein aggregate reduction method comprises administering to the subject an effective amount of a pharmaceutical composition comprising a specific Sentrin-specific protease 1 (SENP-1) inhibitor. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. The composition may comprise the specific SENP-1 inhibitor in an amount ranging from 1 mg to 100 g. The region of the brain may comprise midbrain, striatum (STR), cortex or a combination thereof. The region of the brain may comprise striatum (STR), substantia nigra compacta (SNc) in midbrain, or a combination thereof. The region of the brain may comprise midbrain. The region of the brain may comprise cortex. The subject may be a human. The subject may have suffered from a neurodegenerative disease. The subject may be predisposed to a neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body, and Parkinson-plus disease.

The protein aggregate reduction method may further comprise blocking mRNA expression of the SENP-1. The protein aggregate reduction method may further comprise administering the pharmaceutical composition to the subject orally. The protein aggregate reduction method may further comprise administering the pharmaceutical composition to the subject at a dose of the specific SENP-1 inhibitor at 1-500 mg per kg of the subject's body weight. The protein aggregate reduction method may further comprise administering the pharmaceutical composition to the subject at a dose of the specific SENP-1 inhibitor at 1-500 mg per kg of the subject's body weight daily for 1-10 weeks.

A method is provided for reducing nuclear translocalization of Sentrin-specific protease 1 (SENP-1) from cytosol of cells in a region of the brain of a subject in need thereof. The SENP-1 nuclear translocalization reduction method comprises administering to the subject an effective amount of a pharmaceutical composition comprising a SENP-1 nuclear translocalization blocker. The composition may comprise the SENP-1 nuclear translocalization blocker in an amount ranging from 1 mg to 100 g. The region of the brain may comprise midbrain, striatum (STR), cortex or a combination thereof. The region of the brain may comprise striatum (STR), substantia nigra compacta (SNc) in midbrain, or a combination thereof. The region of the brain may comprise midbrain. The region of the brain may comprise cortex. The subject may be a human. The subject may have suffered from a neurodegenerative disease. The subject may be predisposed to a neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body, and Parkinson-plus disease.

The SENP-1 nuclear translocalization reduction method may further comprise blocking mRNA expression of the SENP-1. The SENP-1 nuclear translocalization reduction method may further comprise administering the pharmaceutical composition to the subject orally. The SENP-1 nuclear translocalization reduction method may further comprise administering the pharmaceutical composition to the subject at a dose of the SENP-1 nuclear translocalization blocker at 1-500 mg per kg of the subject's body weight. The SENP-1 nuclear translocalization reduction method may further comprise administering the pharmaceutical composition to the subject at a dose of the SENP-1 nuclear translocalization blocker at 1-500 mg per kg of the subject's body weight daily for 1-10 weeks.

### BRIEF DESCRIPTION OF THE DRAWINGS

FIGS. 1A-1B show that, in Western blot analysis, the level of SENP-1 was substantially enhanced by MPP+ exposure in N27 parental cells within 24 hrs. A) The examples of Western blots indicate that SENP-1 expression was substantially up-regulated by PD-inducing agent, MPP+. B) The level of SENP-1 was substantially increased by MPP+ treatment, whereas other SENPs, such as SENP-3, -5, -6, -7 and -8 were not drastically affected by MPP+. Student's t-test was applied for significance between vehicle and MPP+, compared to no MPP+ (dark bars) (n=6/group). *: p<0.05, **: p<0.01 and n.s.: not significant.

FIGS. 2A-2E show that SENP-1 was predominantly localized in nuclear fraction and its level was up-regulated by preformed fibrils of alpha-synuclein (PFF) in Western blot analysis. A) In Western blots, SENP-1 in nucleus was also up-regulated by PFF treatment, but SENP-3 was slightly down-regulated. B) The level of SENP-1 was enhanced by PFF exposure in nucleus (Nu), compared to PBS control. C) The level of SENP-3 was reduced by PFF treatment. D) The level of SUMO-1 in cytosol (Cy) was reduced by PFF treatment. E) The level of SUMO-2/3 was not significantly affected by PFF exposure. Total 10 μg of PFF was exposed to N27p cells for 48 h. The cellular Fractionation was performed using a kit (78833; Thermo). Student's t-test was applied for significance, compared to no PFF (dark bars) (n=6/group). *: p<0.05, **: p<0.01 and n.s.: not significant.

FIGS. 3A-3B show that the level of SENP-1 was increased by PFF exposure, while the level of SENP3 was decreased in primary cortical neuron culture. Those neurons were exposed to PFF treatment (5 μg) for 12 h for Western Blot analysis (n=6/group). A) In Western blots, the level of SENP-1 was enhanced by PFF treatment, whereas SENP-3 was slightly reduced by PFF in primary cortical neurons. B) The level of SENP-1 was significantly increased by PFF, whereas SENP-3 was decreased, which are associated with reduced SUMO-1 level and increased SUMO-2/3 level. The level of alpha-synuclein is substantially higher with PFF treatment, mainly due to extra alpha-synuclein from PFF. Student's t-test was applied for significance, compared to no PFF (dark bars) (n=6/group). *: p<0.05, **: p<0.01, ***: p<0.001 and n.s.: not significant.

FIGS. 4A-4G show that the knock-down of SENP-1 using siRNA increases the overall level of SUMO-1 in Western blot analysis. A) The RNA interference suppressed the expression level of SENP-1, which enhanced SUMO-1 level. The SENP-1 siRNA was treated to N27p cells overnight after 48 h of PFF exposure (n=3-4/group), which significantly reduced the level of SENP-1 (B), enhanced the level of SUMO-1 (C), but did not make it different in alpha-synuclein level (D). In Western blots (E), a SENP-1 inhibitor, Momordin reduced the level of SENP-1 (F), which is associated with an increased level of SUMO-1 (G). One-Way ANOVA, Dunnett's test was applied for statistical analysis in comparison with PFF only (#). *: p<0.05, **: p<0.01, p<0.001 and n.s.: not significant.

FIGS. 5A-5F show that, in LDH assays, various SENP-1 inhibitors reduce the cytotoxicity induced by PFF treatment. Several SENP-1 inhibitors, such as SUMO-2 Aldehyde (A), Momordin lc (B), Streptonigrin (C), Hinokiflavone (E), as well as siRNA (F) significantly reduced the cytotoxicity derived from PFF, except oleanolic acid (D). One-way ANOVA, Dunnett's Test, compared to PFF treatment only control (#) (n=4/group). **: p<0.01, ***: p<0.001 and ****: p<0.0001.

FIGS. 6A-6F show that, in MTT assays, SENP-1 inhibitors enhance the cell viability from PFF-mediated toxicity. Several SENP-1 inhibitors, such as SUMO-2 Aldehyde (A), Momordin lc (B), Streptonigrin (C), Hinokiflavone (E), as well as siRNA (F), significantly enhanced the cell viability from the damage derived from PFF, except oleanolic acid (D). One-way ANOVA, Dunnett's Test was applied, compared to PFF treatment only control (#) (n=4/group). *: p<0.05, **: p<0.01, ***: p<0.001 and ****: p<0.0001.

FIGS. 7A-7D show that various SENP-1 inhibitors reduce the levels of protein aggregation and ROS, induced from PFF treatment. N27 cell images labeled for protein aggregates in Thioflavin-T staining (A) and ROS (B) are displayed to show the contrast of protein aggregation level and ROS intensity between SENP-1 inhibitors and PFF only. C) All the SENP-1 inhibitors such as SUMO-2 aldehyde, Momordin lc, Hinokiflavone and Streptonigrin reduced the level of ROS, except Oleanolic acid. D) In immunocytochemistry, the level of ROS was elevated by PFF treatment, which was reversed by various SENP-1 inhibitors. One-way ANOVA, Dunnett's Test (n=10/group) was applied, compared to PFF treatment only control (#). **: p<0.01, ***: p<0.001, ****: p<0.0001 and n.s.: not significant.

FIGS. 8A-8D show that the siRNA of SENP-1 treatment reduces the levels of protein aggregation and ROS, induced from PFF treatment. In immunocytochemistry, N27 cell images labeled for protein aggregates in Thioflavin-T staining (A) and ROS (C) are displayed to show the contrast of protein aggregation level and ROS intensity between siRNA of SENP-1 and NC-1 control or PFF only. B) The siRNA of SENP-1 significantly reduced the level of protein aggregation in Thioflavin-T, compared to PFF only (#) control. D) The level of ROS was elevated by PFF treatment, which was reversed by siRNA of SENP-1. One-way ANOVA, Dunnett's Test (n=10/group). *: p<0.05, ***: p<0.001, ****: p<0.0001 and n.s.: not significant.

FIGS. 9A-9D show that the PFF injection to young mice (3 month-old) increases the level of SENP-1, but slightly decreases SENP-3 in the ventral midbrain (MB), which is correlated with a decrease of SUMO-1 and an increase of SUMO-2/3. A) In Western blot analysis (n=6/group), PFF injection enhanced the level of SENP-1 in the ventral MB. B) PFF injection significantly increased the level of SENP-1, while it reduced the level of SENP-3 in the ventral MB. C) In Western blot, the PFF effects on SENP-1 (or SUMO-1) or SENP-3 (or SUMO-2/3) were not significantly detected in the STR (D). Student's t-test was applied for significance. *: p<0.05 and **: p<0.01. n.s.: not significant.

FIGS. 10A-10B show that the level of SENP-1 is higher in human PD SNpc than that in age-& gender-matched controls, while the level of SENP-3 is lower in human PD midbrain than that in controls, which is correlated with a decrease of SUMO-1 and an increase of SUMO-2/3 in PD SNpc tissues. A) In Western blots, the level of SENP-1 was consistently higher in human PD SNpc tissues than that in control (n=8/group). B) In Western blot analyses, the level of SENP-1 was elevated, while SENP3 was lower in PD brains, which are corelated with lower level of SUMO-1 and higher level of SUMO-2/3 in PD brains, compared to age-matched controls. Student's t-test was applied for significance. **: p<0.01, ****: p<0.0001 and n.s.: not significant.

FIGS. 11A-11G show that a SENP-1 inhibitor, Momordin lc treatment was effective in improving mobility in PFF-injected old mice (>12 month-old). A) An experimental schematic shows time points for PFF injection, oral gavage of Momordin, behavioral tests, and following brain isolation. In behavioral tests, 10 and/or 50 mg/kg of oral Momordin treatment enhances the mobility on rotarod (B), improves the nesting (C) and grooming (D), reduces hindlimb clasping (E) and turn time (F), as well as climb-down time (G) in pole tests. One-way ANOVA, Dunnett's test was used, compared with PFF only (#) control (n=7-8/group). *: p<0.05, **: p<0.01, ***p<0.001, ****: p<0.0001 and n.s.: not significant.

FIGS. 12A-12D show that, in immunohistochemical analyses, Momordin treatment prevents and/or reverses the damage from the PFF-induced toxicity in the striatum (STR) and the substantia nigra compacta (SNc) (n=7/group). A) In the striatal sections, the intensity of TH+ neurons was recovered by the treatment from PFF-induced damage in the STR. B) In the SNc region, TH+ labeled dopaminergic neuronal cell body was significantly recovered from PFF damage with both doses of Momordin treatment (10 and 50 mg/kg). In immunohistochemical analyses, the oral treatment of Momordin for 6-7 weeks improves the intensity of TH+ label in the striatum (C) and the number of dopaminergic neurons in the SNc (D) (n=7/group). One-way ANOVA, Dunnett's test was applied to compare with PFF only (#) control. **: p<0.01 and p<0.0001.

FIGS. 13A-13C show that, in immunohistochemical analyses, both 10 and 50 mg/kg Momordin treatment reduce the levels of phosphorylated alpha-synuclein and protein aggregates in Thioflavin-T from PFF-induced toxicity in the striatum. A) In the striatal sections, oral Momordin treatment alleviated both phosphorylated form of alpha-synuclein (red) and protein aggregation (green) in the measurements of fluorescence intensity. In the striatum of PFF-injected mice, both doses of Momordin treatment significantly reduced the levels of phospho-α-synuclein (B) as well as protein aggregates (C) in a dose-dependent manner (n=7/group). One-way ANOVA, Dunnett's test was applied to compare with PFF only (#) control. ***: p<0.001 and ****: p<0.0001.

FIGS. 14A-14C show that, in immunohistochemical analyses, both 10 and 50 mg/kg Momordin treatment reduce the levels of phosphorylated alpha-synuclein and protein aggregates in Thioflavin-T from PFF-induced toxicity in the SNc. A) In the ventral midbrain sections, oral Momordin treatment reduced the levels of phosphorylated form of alpha-synuclein (red) and protein aggregation (green) in the measurements of fluorescence intensity. In the SNc of PFF-injected mice, both doses of Momordin treatment significantly reduced the levels of phospho-α-synuclein (B) as well as protein aggregates (C) in a dose-dependent manner (n=7/group). One-way ANOVA, Dunnett's test was applied to compare with PFF only (#) control. ***: p<0.001 and **: p<0.0001.

FIGS. 15A-15G show that, in Western blot analysis using tissue extracts, Momordin treatment reduces the levels of phosphorylated alpha-synuclein and SENP-1, which are associated with the elevated level of SUMO-1 in the midbrain and striatum. A) The images of WB show that Momordin reversed the toxicity from PFF injection in reducing phospho-α-synuclein and SENP-1 in brains. The PFF-induced SENP-1 was reduced by Momordin in the MB (B) and STR (C), which is linked to the enhanced level of SUMO-1 in the MB (D) and the STR (E). The pathological marker, phosphorylated α-synuclein was increased by PFF injection, which was reversed by both doses of Momordin treatment in the MB (F) and the STR (G). Results were displayed in mean±SEM (n=7-8/group) and analyzed using One-way ANOVA, Dunnett's test in comparison with PFF only (#) control. **: p<0.01, ***: p<0.001, ****: p<0.0001 and n.s.: not significant.

FIGS. 16A-16G show that, using α-synuclein IP tissue samples, Momordin reduces the levels of phosphorylated alpha-synuclein and SENP-1 on α-synuclein, which are associated with the elevated level of SUMO-1 on α-synuclein from the MB and STR. A) In Western blots, Momordin reversed the toxicity from PFF injection in reducing phospho-α-synuclein and SENP-1 on α-synuclein in the MB and STR. The PFF-induced SENP-1 was reduced by Momordin in the MB (B) and STR (C), which is followed by an increase in SUMO-1 conjugated α-synuclein in the MB (D) and the STR (E). A pathological marker, phosphorylated α-synuclein was increased by PFF injection, which was reversed by Momordin treatment in the MB (F) and the STR (G). Results were displayed in mean #SEM (n=7-8/group) and analyzed using One-way ANOVA, Dunnett's test in comparison with PFF only (#) control. *: p<0.05, **: p<0.01, ***: p<0.001, ****: p<0.0001 and n.s.: not significant.

FIGS. 17A-17B show the locations of nuclear localization sequence (NLS) of human SENP-1 and point mutation sites for blocking SENP-1 nuclear translocalization from the cytosol. A) The human SENP-1 includes TAG, NLS1, NLS2, NLS3 and core domain. B) The critical domain of SENP-1 for blocking nuclear translocation is NLS1, in which the normal WT sequence as well as two double point-mutations (Mutation 01 and Mutation 02) are displayed as single amino acid character symbols. Abb: P: proline, K: lysine, T: threonine, Q: glutamine, R: arginine, and G: glycine. Numbers indicate the location of each amino acid in the amino acid sequence of SENP-1.

FIGS. 18A-18B show that PFF-induced cell damage was alleviated in N27 rat dopaminergic cells overexpressing SENP-1 mutant 1 (M1) or mutant 2 (M2), compared to control cells overexpressing vector only (Veh) or wild-type SENP-1 (WT). A) In the cell viability (MTT) assay, exposure to 1 μg/ml PFF for 24 hrs resulted in reduced cell viability in WT overexpression, not in SENP-1 nuclear trans-localization mutants (M1 or M2) overexpression, as compared with Veh only. B) In the cytotoxicity (LDH) assay, the overexpression of WT SENP-1 effectively mediated PFF-induced toxicity, whereas nuclear trans-localization mutants (M1 & M2) failed to mediate the PFF-induced cytotoxicity. The results are displayed in mean #SEM (n=7-8/group) based on analysis using One-way ANOVA, Dunnett's test in comparison with PFF only (#) control. **: p<0.01.

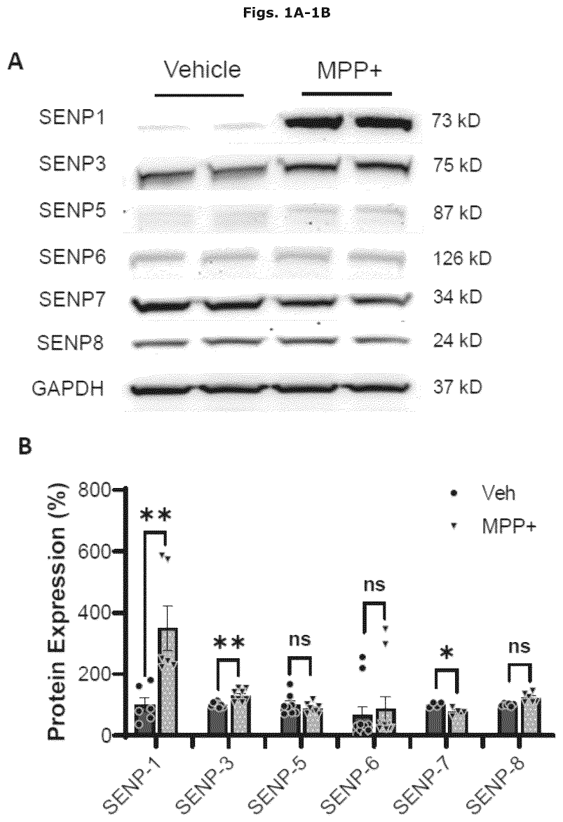

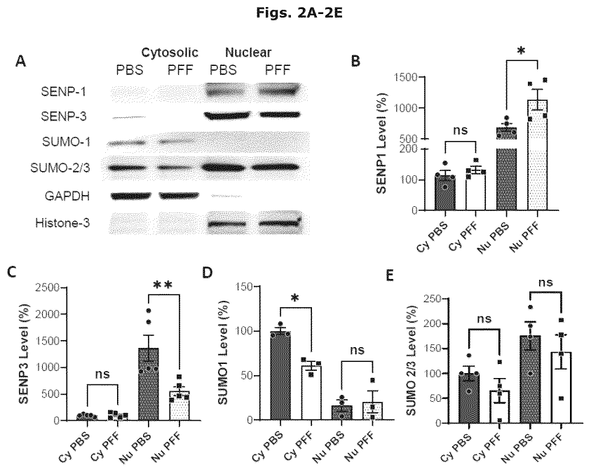

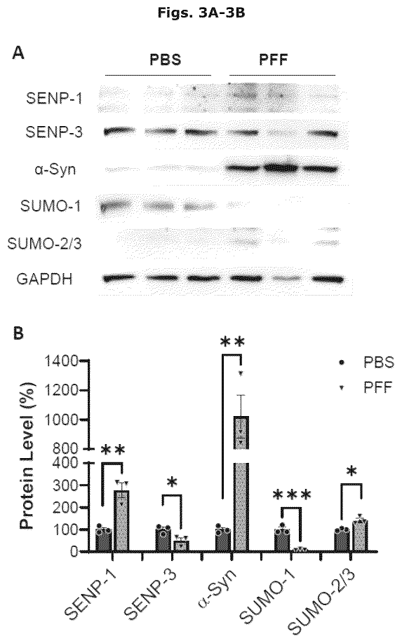

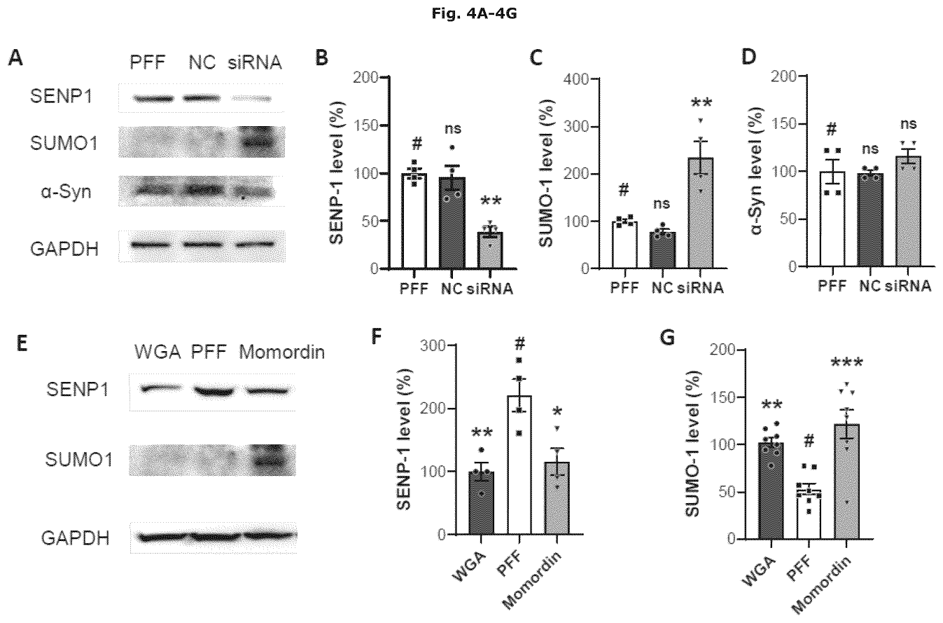

### DETAILED DESCRIPTION OF THE INVENTION

The present invention provides methods for treating or preventing a neurodegenerative disease in a subject with a Sentrin-specific protease 1 (SENP-1) inhibitor and compositions comprising the SENP-1 inhibitor for treating or preventing the neurodegenerative disease in the subject. The invention is based on the inventor's surprising discovery of a lower level of Small Ubiquitin-like modifier (SUMO)-1 associated with the pathological conditions of PD in mouse models, including MPTP- or PFF-injected mice as well as alpha-synuclein wild-type overexpressing transgenic mice. The inventor has also discovered that the inhibition of SUMO-1 removing enzyme activity, SUMO protease (SENP-1) from target proteins, including alpha-synuclein, is beneficial in inhibiting detrimental protein aggregation and further oxidative stress in the pathological processes of Parkinson's disease, Dementia with Lewy body (DLB) or other neurodegenerative diseases. Although targeting SENP-1 inhibition has been conceptualized and applied to suppress tumor or metastasis in cancer research, the inventor has unexpectedly discovered that SENP inhibition is effective for treating or preventing neurodegenerative diseases, and chronic administration of a specific SENP-1 inhibitor in a moderate dose as a therapeutic inhibits the onset and/or the rate of progression and/or severity of one or more symptoms of Parkinson's disease and/or of a Parkinson-plus condition. The inventor has also contemplated that a chronic moderate therapeutic dose can be used in conjunction with certain other agents (e.g., L-Dopa/carbidopa and/or derivatives thereof) to achieve improved efficacy in treatment or prophylaxis of Parkinson's disease, a Parkinson-plus condition or other neurodegenerative disorders.

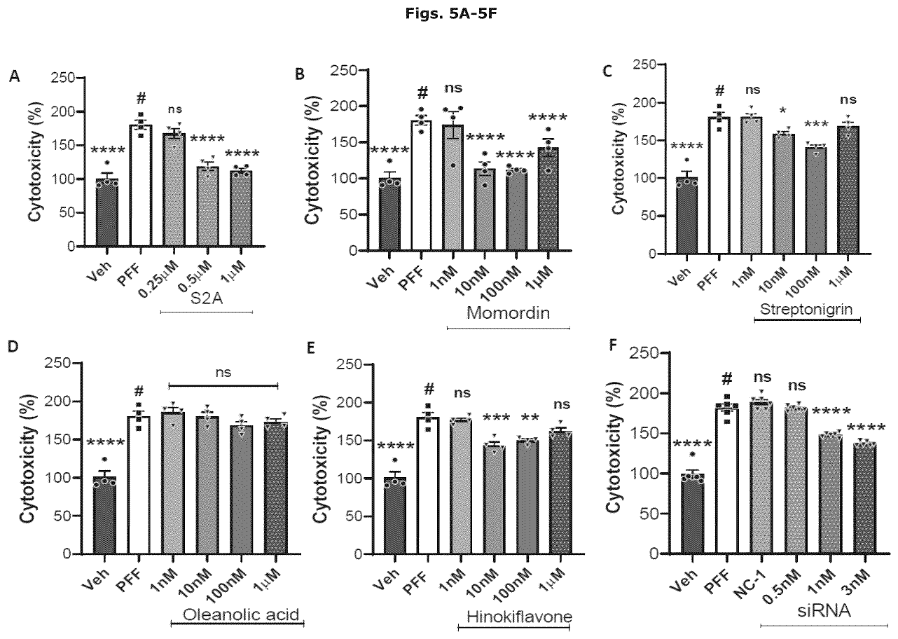

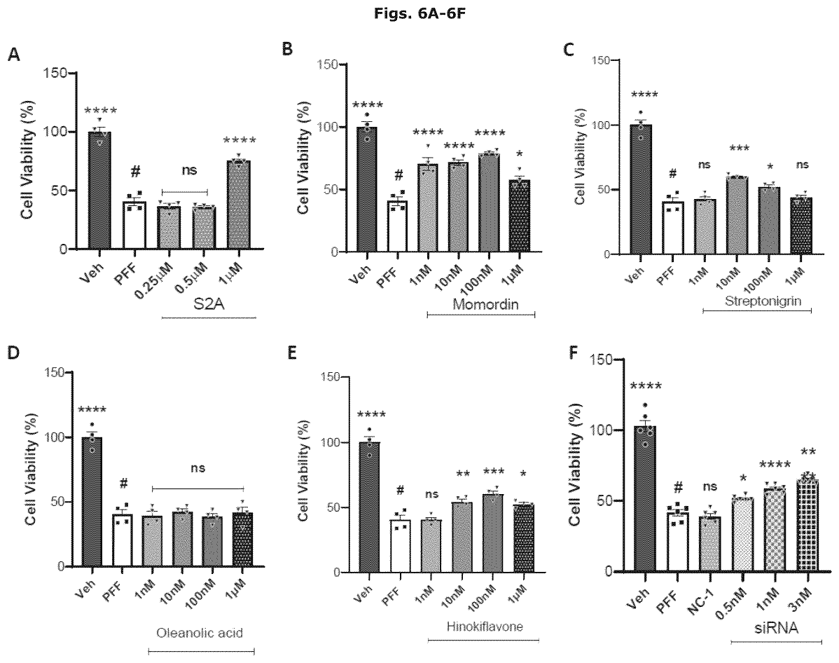

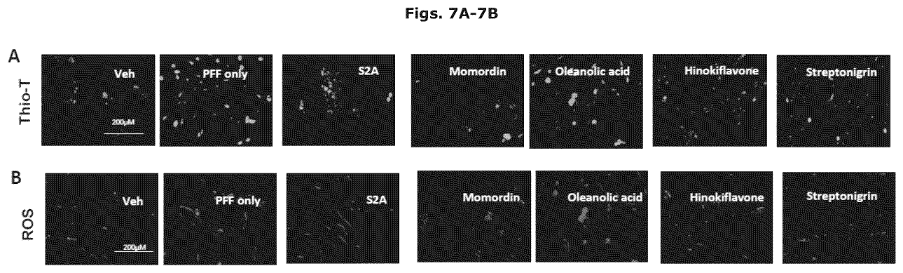

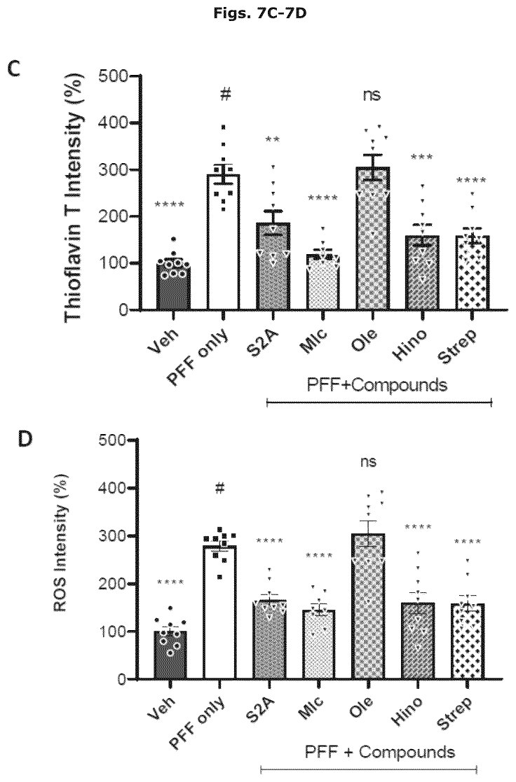

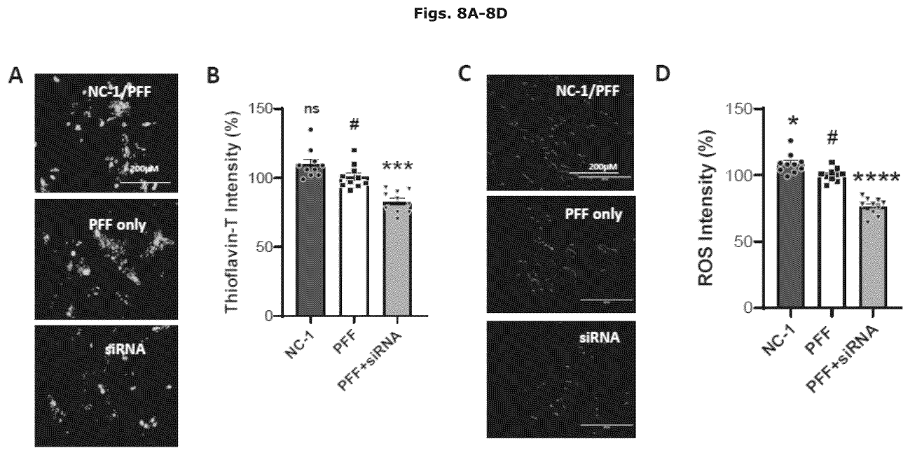

The inventor has developed methods of slowing or inhibiting onset or severity of one or more symptoms associated with Parkinson's Disease (PD) and/or a “Parkinson-plus” disease, for example, involving oral administration of SENP-1 inhibitors (e.g., Momordin lc) as a novel approach to halt PD progression and, to some extent, reverse the pathological progress. In a study, numerous commercially available SENP-1 inhibitors were screened for their recovery effects as evidenced by preformed fibrils of alpha-synuclein (PFF)-induced PD models in vitro. The screening process included several specific SENP-1 inhibitors (e.g., SUMO-2 Aldehyde, Momordin lc, streptonigrin, hinokiflavone, and siRNAs of SENP-1), and non-specific SENP-1 inhibitors (e.g., oleanolic acid, and gallic acid) in N27 dopaminergic cells after exposure to PFF for 24 hrs. Using well-established methods, such as cytotoxicity (LDH) or cell viability (MTT) assay, and measurement of Reactive Oxygen Species (ROS) and protein aggregation in Thioflavin-T staining, the inventor has unexpected discovered that specific SENP-1 inhibitors (e.g., SUMO-2 Aldehyde, Momordin lc, streptonigrin, hinokiflavone, and SiRNAs of SENP-1) were clearly efficacious not only in preventing protein aggregation-mediated damage, but also in reversing PFF-induced damage to nearly healthy or functional state of dopaminergic cells (FIGS. 5-8).

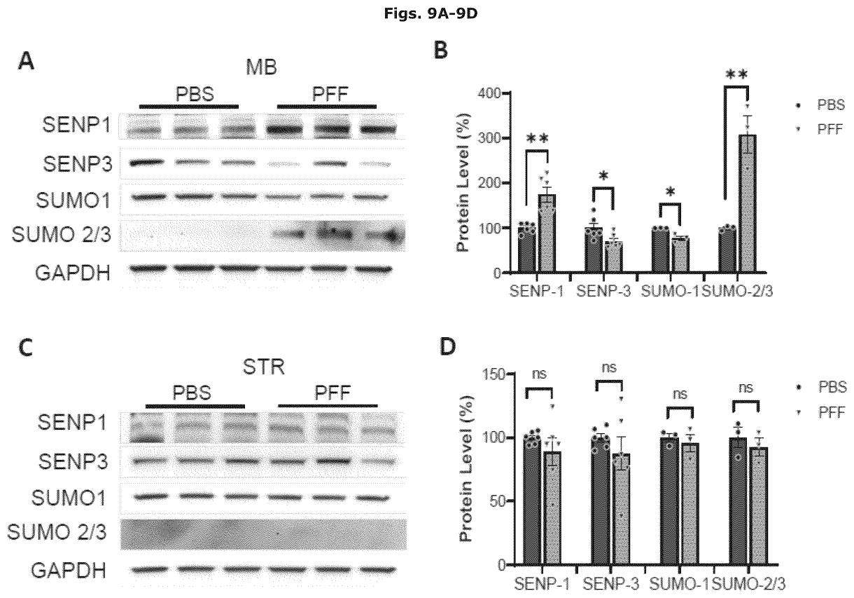

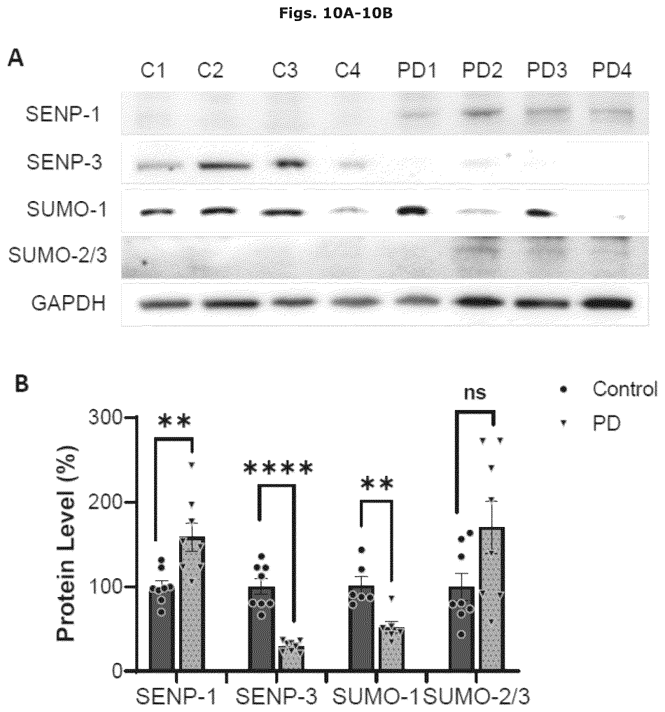

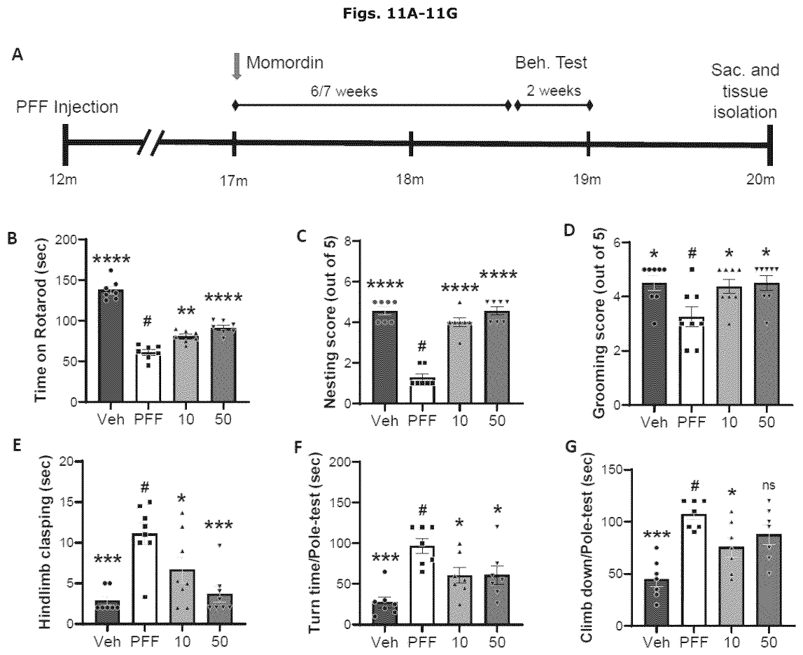

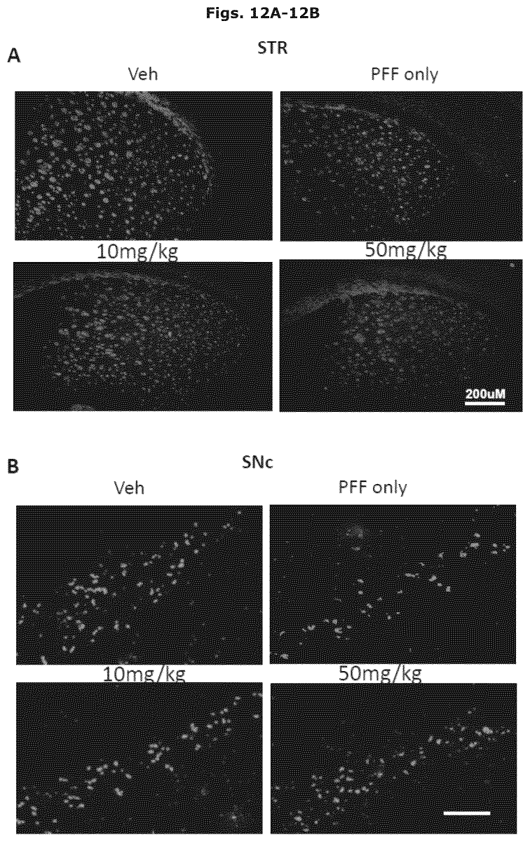

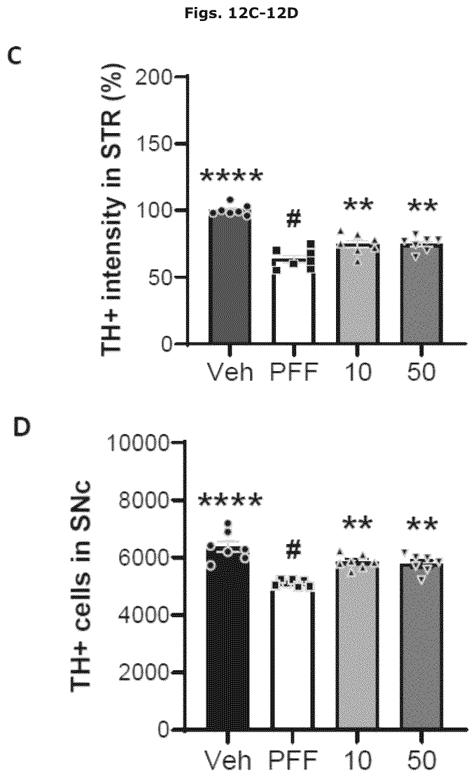

In particular, the inventor has discovered that Momordin lc not only reduced the level of alpha-synuclein accumulation/aggregation in Thioflavin-T staining from PFF injection in various brain regions, but also alleviated the level of ROS in nigrostriatal neurons and further to reverse dopaminergic neuronal death in mouse brains (FIG. 12). Importantly, Momordin lc at both low and high dosages (10 or 50 mg/kg) was shown to be safe in mice and effectively pass the BBB for efficacy. PFF toxicity was found to enhance the expression level of SENP-1 in the midbrain, and the enhanced expression level of SENP-1 was associated with a lower level of SUMO-1 in SNpc (SNc in mice) as compared with other regions in the midbrain (FIGS. 9 and 12). The up-regulation of the SENP-1 expression in a human midbrain was also found associated with PD pathology since the SENP-1 expression level in SNpc from human PD patients was consistently higher than age-& gender-matched controls (n=8/group) (FIG. 10). In an in vivo study, 10 or 50 mg/kg oral Momordin lc treatment improved the behavioral deficits derived from PFF injection (FIG. 11), which was supported by immunohistochemical analyses. The oral treatment of PFF-injected mice with 50 mg/kg Momordin for 6-7 weeks was found effective for the treated mice to recover from damages in the striatum and Substantia Nigra compacta (SNc) (n=7-8/group) (FIG. 12).

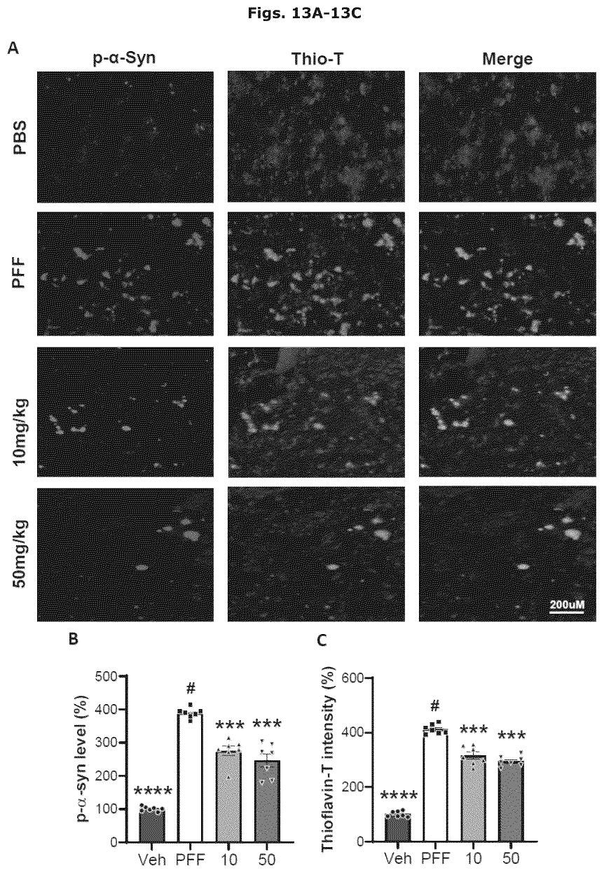

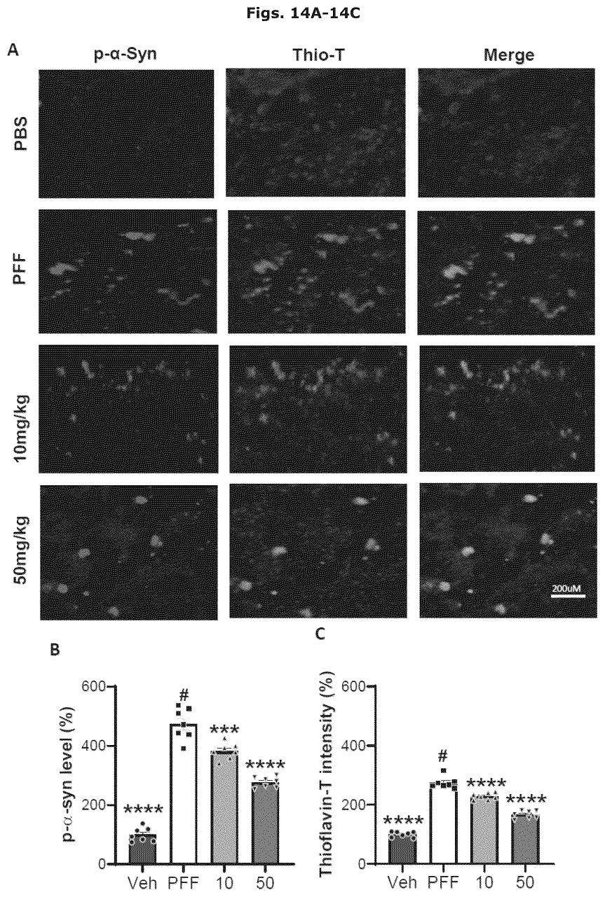

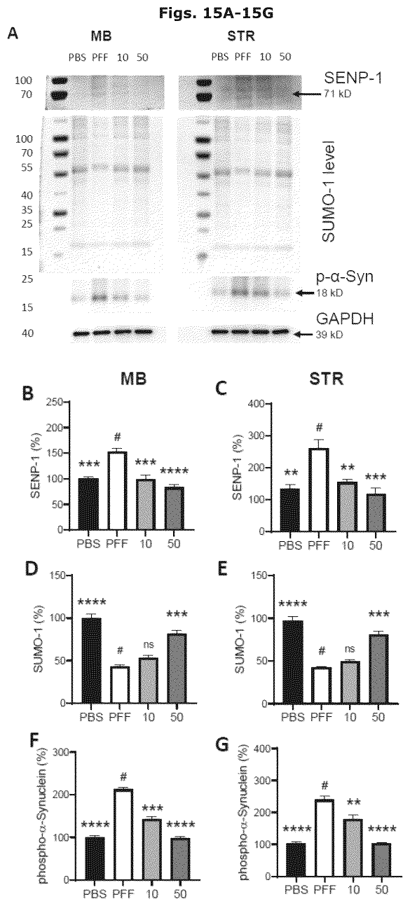

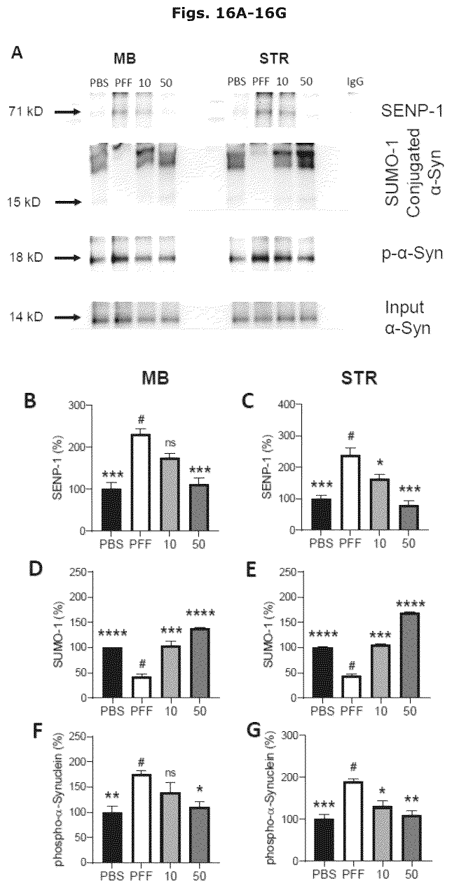

The inventor has also discovered that chronic oral administration of a SENP-1 inhibitor prevents both oxidative stress-induced alpha-synuclein accumulation and neuronal cell death in PD models at a dosage of 10 or 50 mg/kg as a therapeutic dose, which is considered below the toxic dosage in mice. Momordin administration was initially found to protect against in vitro cell death induced by MPP+ or PFF-mediated toxic conditions using N27 dopaminergic cell line. Oral Momordin administration (10 or 50 mg/kg) to 15-16 months old mice for 6-7 weeks starting 3-4 months after PFF injection into the dorsal striatum was effective to prevent the accumulation of phosphorylated alpha-synuclein (toxic form) in multiple brain regions, previously shown to occur as a consequence of dopaminergic neuronal loss, mimicking PD-related signs in mice (FIGS. 13-16). The treatment alleviates severe motor deficits in behavioral tests, for example, rotarod, nesting, grooming, hindlimb clasping and pole tests (FIG. 11).

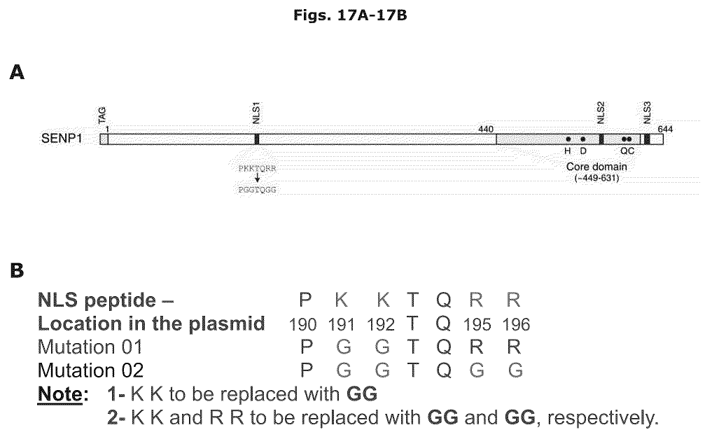

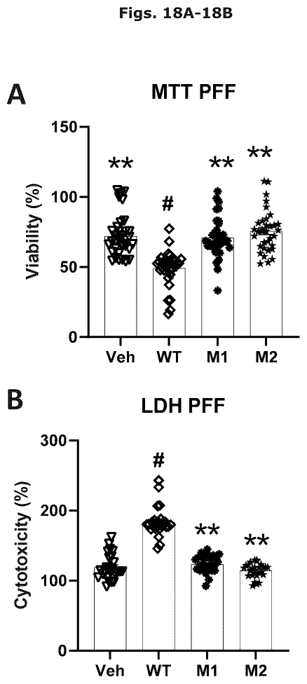

Our findings also suggest that SENP-1 WT overexpression-mediated toxicity is at least in part derived from the nuclear translocalization of SENP-1, since SENP-1 is predominantly detected in the nucleus, but minimally detected in cytosol. In the PFF treated cell model, two different double point mutations of SENP-1 (Mutation 01 and Mutation 02) (FIGS. 17A-17B) were crucial for blocking nuclear translocalization, by which PFF-mediated cytotoxicity was alleviated (FIGS. 18A-18B), suggesting that the SENP-1 toxicity is critically required for the translocation of SENP-1 into the nucleus, which mediates cell death. Thus, blocking the translocalization of SENP-1 into nucleus may be a critical therapeutic target for preventing or reversing neurodegenerative pathology.

The term “specific Sentrin-specific protease 1 (SENP-1) inhibitor” as used herein refers to an agent capable of interrupting or reducing the activity and/or expression of SENP-1, but not unrelated targets. Examples of the specific SENP-1 inhibitors include small ubiquitin-like modifier 2 (SUMO-2) aldehyde, momordin lc, streptonigrin, hinokiflavone and siRNAs of SENP-1. The agent may be a biological molecule, a chemical compound or a combination thereof. The biological molecule may be a protein or nucleic acid (e.g., DNA or RNA).

The term “non-specific Sentrin-specific protease 1 (SENP-1) inhibitor” as used herein refers to an agent capable of interrupting or reducing the activity of unrelated targets, including SENP-1. Examples of the non-specific SENP-1 inhibitors include oleanolic acid and gallic acid. The agent may be a biological molecule, a chemical compound or a combination thereof. The biological molecule may be a protein or nucleic acid (e.g., DNA or RNA).

The term “Sentrin-specific protease 1 (SENP-1) nuclear translocalization blocker” as used herein refers to an agent capable of interrupting or reducing translocalization of the SENP-1 from the cytosol to a nucleus in a cell. The agent may be a biological molecule, a chemical compound or a combination thereof. The biological molecule may be a protein or nucleic acid. The nucleic acid may be a DNA or RNA. The nucleic acid may be a DNA or RNA. The RNA may be siRNA, RNAi, anti-sense RNA or a combination thereof. The SENP-1 nuclear translocalization may be detected by Western blot or ELISA, using nuclear fractions or isolates with or without SENP-1 immunoprecipitation.

The term “neurodegenerative disease” as used herein refers to a disease or disorder involving loss of biologically active neurons in the central nervous system of a subject. Such neuronal loss may be associated with aging and/or mediated by protein aggregation. Examples of the neurodegenerative disease include Parkinson's disease, Alzheimer's disease, Huntington's disease, Amyotrophic Lateral Sclerosis, Spinal muscular atrophy, Friedreich ataxia, Dementia with Lewy body, and Parkinson-plus disease. The Parkinson-plus disease may be multiple system atrophy (MSA), progressive supranuclear palsy (PSP), corticobasal degeneration (CBD), and other Lewy body diseases.

The term “symptom associated with a neurodegenerative disease” as used herein refers to directly related to the type of neuronal loss, which is associated with a particular dysfunction in the Central Nervous System. Exemplary symptoms associated with a neurodegenerative disease include memory loss, forgetfulness, cognitive decline, apathy, anxiety, agitation, smell loss (anosmia), hallucination, sleep disruption, voice changes, depression, mood changes, and abnormal bowel movements (constipation/diarrhea).

The term “subject” as used herein refers to a mammal. The subject may be a human or a non-human primate. The subject may be a domesticated mammal (e.g., canine or feline), a laboratory mammal (e.g., mouse, rat, rabbit, hamster, and guinea pig), or an agricultural mammal (e.g., equine, bovine, porcine, and ovine). Where the subject is a human. The human may be an adult male, adult female, adolescent male, adolescent female, male child, or female child. The human may be under the care of a physician or other healthcare worker in a hospital, psychiatric care facility, as an outpatient, or other clinical context. The subject may not be under the care or prescription of a physician or other health worker.

The subject may not have been treated for a neurodegenerative disease. The subject may have been treated for a neurodegenerative disease with, for example, L-Dopa/carbidopa (Sinemet), dopamine agonist, MAO-B inhibitors, COMT inhibitors, Amantadine, Anticholinergics, Donepezil, Galantamine, Memantine, Rivastigmine, Namzaric and Adenosine A2a antagonists.

The term “Serine 129 phosphorylated alpha-synuclein” used herein refers to alpha-synuclein, in which the Serine residue at position 129 is phosphorylated.

The term “effective amount” as used herein refers to an amount of a composition comprising a specific SENP-1 inhibitor required to achieve a stated goal (e.g., treating or preventing a neurodegenerative disease in a subject in need thereof, increasing neurons or small ubiquitin-like modifier 1 (SUMO-1) in a region of the brain of a subject, or reducing Sentrin-specific protease 1 (SENP-1), Serine 129 phosphorylated alpha-synuclein or protein aggregates in a region of the brain of a subject). The effective amount of the pharmaceutical composition comprising a specific SENP-1 inhibitor may vary depending upon the stated goals, the physical characteristics of the subject, the nature and severity of the neurodegenerative disease, the existence of related or unrelated medical conditions, the nature of the specific SENP-1 inhibitor, the pharmaceutical composition comprising the specific SENP-1 inhibitor, the means of administering the pharmaceutical composition to the subject, and the administration route. A specific dose for a given subject may generally be set by the judgment of a physician.

The term “mitigating” as used herein refers to reduction or elimination of severity a symptom associated with a disease (e.g., neurodegenerative disease). The reduction may be at least about 10%, 20%, 30%, 40%, 50%, 60%, 70%, 80%, or 90%.

The term “onset” as used herein refers to the beginning of a change in cells (e.g., neurons), biological molecules (e.g., small ubiquitin-like modifier 1 (SUMO-1), Sentrin-specific protease 1 (SENP-1), Serine 129 phosphorylated alpha-synuclein, and protein aggregates), or a symptom associated with a disease (e.g., neurodegenerative disease). The change may be at least about 10%, 20%, 30%, 40%, 50%, 60%, 70%, 80%, or 90%.

The term “administering” as used herein refers to local and/or systemic administration, for example, enteral and parenteral administration. Routes of administration include oral administration, administration as a suppository, topical contact, intravenous administration, intraperitoneal administration, intramuscular administration, intralesional administration, nasal administration, subcutaneous administration, the implantation of a slow-release and/or regulated release device, for example, a mini-osmotic pump, a depot formulation, to a subject. Administration may be by any route, for example, a parenteral or transmucosal (e.g., oral, nasal, vaginal, rectal, or transdermal) route. Parenteral administration includes intravenous, intramuscular, intra-arteriole, intradermal, subcutaneous, intraperitoneal, intraventricular, ionophoretic and intracranial administration. Other modes of delivery may include the use of liposomal formulations, intravenous infusion, or transdermal patches.

The terms “systemic administration” and “systemically administered” are used herein interchangeably and refer to a method of administering an active ingredient (e.g., a SENP-1 inhibitor) to a subject such that the active ingredient is delivered to sites in the subject, including a targeted site of a pharmaceutical action involving the active ingredient via, for example, a circulatory system. The systemic administration includes oral, intranasal, rectal and parenteral (i.e., other than through an alimentary tract, such as intramuscular, intravenous, intra-arterial, transdermal and subcutaneous) administration.

The present invention provides a pharmaceutical composition. The pharmaceutical composition may comprise a specific SENP-1 inhibitor in an amount effective for:
(a) treating a neurodegenerative disease in a subject in need thereof;
(b) preventing a neurodegenerative disease in a subject in need thereof;
(c) increasing neurons in a region of the brain of a subject in need thereof;
(d) increasing small ubiquitin-like modifier 1 (SUMO-1) in a region of the brain of a subject in need thereof;
(e) reducing Sentrin-specific protease 1 (SENP-1) in a region of the brain of a subject in need thereof;
(f) reducing Serine 129 phosphorylated alpha-synuclein in a region of the brain of a subject in need thereof; and/or
(g) reducing protein aggregates in a region of the brain of a subject.

According to the pharmaceutical composition of the present invention, the specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. For example, the specific SENP-1 inhibitor is momordin lc. The pharmaceutical composition may comprise the specific SENP-1 inhibitor in an amount ranging from 1 mg to 100 g, from 1 mg to 1 g or from 1 mg to 500 mg.

The pharmaceutical composition may comprise a SENP-1 nuclear translocalization blocker in an amount effective for reducing nuclear translocalization of SENP-1 from cytosol of cells in a region of the brain of a subject in need thereof. The pharmaceutical composition may comprise the SENP-1 nuclear translocalization blocker in an amount ranging from 1 mg to 100 g, from 1 mg to 1 g or from 1 mg to 500 mg.

According to the pharmaceutical composition of the present invention, the subject may be human. The subject may be a male or female. The subject may be an adult. The subject may be 20, 25, 30, 35, 40, 45, 50, 55, 60, 65, 70, 75 or 80 years of age or older. The subject may have suffered from a neurodegenerative disease. The subject may or may not have been treated for the neurodegenerative disease. The subject may be predisposed to a neurodegenerative disease.

According to the pharmaceutical composition of the present invention, the neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease. The Parkinson-plus disease may be selected from the group consisting of multiple system atrophy (MSA), progressive supranuclear palsy (PSP), corticobasal degeneration (CBD), and other Lewy body diseases.

According to the pharmaceutical composition of the present invention, the region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex.

The pharmaceutical composition may comprise a specific SENP-1 inhibitor in an amount effective for treating a neurodegenerative disease in a subject in need thereof. The specific SENP-1 inhibitor may be in an amount effective for mitigating or reversing severity of a symptom associated with the neurodegenerative disease. The symptom may be selected from the group consisting of memory loss, forgetfulness, cognitive decline, apathy, anxiety, agitation, smell loss (anosmia), hallucination, sleep disruption, voice changes, depression, mood changes, and abnormal bowel movements (constipation/diarrhea). The specific SENP-1 inhibitor may be in an amount effective for improving mobility of the subject. The mobility may be selected from the group consisting of bradykinesia (e.g., limb stiffness), tremor, dyskinesia (e.g., uncontrollable movements), postural instability, nesting, grooming, hindlimb clasping, pole-test, turn time, climb-down time, cross-beam, nesting and rotarod. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. For example, the specific SENP-1 inhibitor is momordin lc. The pharmaceutical composition may comprise the specific SENP-1 inhibitor in an amount ranging from 1 mg to 100 g, from 1 mg to 1 g or from 1 mg to 500 mg. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease. The Parkinson-plus disease may be selected from the group consisting of multiple system atrophy (MSA), progressive supranuclear palsy (PSP), corticobasal degeneration (CBD), and other Lewy body diseases. The subject may be a human. The subject may be 20, 25, 30, 35, 40, 45, 50, 55, 60, 65, 70, 75 or 80 years of age or older. The subject may have suffered from a neurodegenerative disease. The subject may or may not have been treated for the neurodegenerative disease.

The pharmaceutical composition may comprise a specific SENP-1 inhibitor in an amount effective for preventing a neurodegenerative disease in a subject in need thereof. The specific SENP-1 inhibitor may be in an amount effective for delaying onset of a symptom associated with the neurodegenerative disease in the subject. The symptom may be selected from the group consisting of memory loss, forgetfulness, cognitive decline, apathy, anxiety, agitation, smell loss (anosmia), hallucination, sleep disruption, voice changes, depression, mood changes, and abnormal bowel movements (constipation/diarrhea). The delay of onset of the symptom associated with the neurodegenerative disease may be determined using a control not receiving the pharmaceutical composition. The specific SENP-1 inhibitor may be in an amount effective for delaying onset of motor dysfunction in the subject. The motor dysfunction may be selected from the group consisting of bradykinesia (e.g., limb stiffness), tremor, dyskinesia (e.g., uncontrollable movements), postural instability, nesting, grooming, hindlimb clasping, pole-test, turn time, climb-down time, cross-beam, nesting and rotarod. The delay of onset of the motor dysfunction may be determined using a control not receiving the pharmaceutical composition. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. For example, the specific SENP-1 inhibitor is momordin lc. The pharmaceutical composition may comprise the specific SENP-1 inhibitor in an amount ranging from 1 mg to 100 g, from 1 mg to 1 g or from 1 mg to 500 mg. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease. The Parkinson-plus disease may be selected from the group consisting of multiple system atrophy (MSA), progressive supranuclear palsy (PSP), corticobasal degeneration (CBD), and other Lewy body diseases. The subject may be a human. The subject may be 20, 25, 30, 35, 40, 45, 50, 55, 60, 65, 70, 75 or 80 years of age or older. The subject may be predisposed to a neurodegenerative disease.

The pharmaceutical composition may comprise a specific SENP-1 inhibitor in an amount effective for increasing neurons in a region of the brain of a subject in need thereof. The neuron may be dopaminergic neurons. The dopaminergic neurons may be positive for Tyrosine hydroxylases (TH+). The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. For example, the specific SENP-1 inhibitor is momordin lc. The pharmaceutical composition may comprise the specific SENP-1 inhibitor in an amount ranging from 1 mg to 100 g, from 1 mg to 1 g or from 1 mg to 500 mg. The subject may be a human. The subject may be 20, 25, 30, 35, 40, 45, 50, 55, 60, 65, 70, 75 or 80 years of age or older. The subject may have suffered from a neurodegenerative disease. The subject may or may not have been treated for the neurodegenerative disease. The subject may be predisposed to a neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease. The Parkinson-plus disease may be selected from the group consisting of multiple system atrophy (MSA), progressive supranuclear palsy (PSP), corticobasal degeneration (CBD), and other Lewy body diseases.

The pharmaceutical composition may comprise a specific SENP-1 inhibitor in an amount effective for increasing small ubiquitin-like modifier 1 (SUMO-1) in a region of the brain of a subject in need thereof. The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. For example, the specific SENP-1 inhibitor is momordin lc. The pharmaceutical composition may comprise the specific SENP-1 inhibitor in an amount ranging from 1 mg to 100 g, from 1 mg to 1 g or from 1 mg to 500 mg. The subject may be a human. The subject may be 20, 25, 30, 35, 40, 45, 50, 55, 60, 65, 70, 75 or 80 years of age or older. The subject may have suffered from a neurodegenerative disease. The subject may or may not have been treated for the neurodegenerative disease. The subject may be predisposed to a neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease. The Parkinson-plus disease may be selected from the group consisting of multiple system atrophy (MSA), progressive supranuclear palsy (PSP), corticobasal degeneration (CBD), and other Lewy body diseases.

The pharmaceutical composition may comprise a specific SENP-1 inhibitor in an amount effective for reducing Sentrin-specific protease 1 (SENP-1) in a region of the brain of a subject in need thereof. The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. For example, the specific SENP-1 inhibitor is momordin lc. The pharmaceutical composition may comprise the specific SENP-1 inhibitor in an amount ranging from 1 mg to 100 g, from 1 mg to 1 g or from 1 mg to 500 mg. The subject may be a human. The subject may be 20, 25, 30, 35, 40, 45, 50, 55, 60, 65, 70, 75 or 80 years of age or older. The subject may have suffered from a neurodegenerative disease. The subject may or may not have been treated for the neurodegenerative disease. The subject may be predisposed to a neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease. The Parkinson-plus disease may be selected from the group consisting of multiple system atrophy (MSA), progressive supranuclear palsy (PSP), corticobasal degeneration (CBD), and other Lewy body diseases.

The pharmaceutical composition may comprise a specific SENP-1 inhibitor in an amount effective for reducing Serine 129 phosphorylated alpha-synuclein in a region of the brain of a subject in need thereof. The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. For example, the specific SENP-1 inhibitor is momordin lc. The pharmaceutical composition may comprise the specific SENP-1 inhibitor in an amount ranging from 1 mg to 100 g, from 1 mg to 1 g or from 1 mg to 500 mg. The subject may be a human. The subject may be 20, 25, 30, 35, 40, 45, 50, 55, 60, 65, 70, 75 or 80 years of age or older. The subject may have suffered from a neurodegenerative disease. The subject may or may not have been treated for the neurodegenerative disease. The subject may be predisposed to a neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease. The Parkinson-plus disease may be selected from the group consisting of multiple system atrophy (MSA), progressive supranuclear palsy (PSP), corticobasal degeneration (CBD), and other Lewy body diseases.

The pharmaceutical composition may comprise a specific SENP-1 inhibitor in an amount effective for reducing protein aggregates in a region of the brain of a subject in need thereof. The protein aggregates may be detected by thioflavin-T. The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. For example, the specific SENP-1 inhibitor is momordin lc. The pharmaceutical composition may comprise the specific SENP-1 inhibitor in an amount ranging from 1 mg to 100 g, from 1 mg to 1 g or from 1 mg to 500 mg. The subject may be a human. The subject may be 20, 25, 30, 35, 40, 45, 50, 55, 60, 65, 70, 75 or 80 years of age or older. The subject may have suffered from a neurodegenerative disease. The subject may or may not have been treated for the neurodegenerative disease. The subject may be predisposed to a neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease. The Parkinson-plus disease may be selected from the group consisting of multiple system atrophy (MSA), progressive supranuclear palsy (PSP), corticobasal degeneration (CBD), and other Lewy body diseases.

The pharmaceutical composition may comprise a SENP-1 nuclear translocalization blocker in an amount effective for reducing SENP-1 nuclear translocalization from cytosol of cells in a region of the brain of a subject in need thereof. The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex. The pharmaceutical composition may comprise the SENP-1 nuclear translocalization blocker in an amount ranging from 1 mg to 100 g, from 1 mg to 1 g or from 1 mg to 500 mg. The subject may be a human. The subject may be 20, 25, 30, 35, 40, 45, 50, 55, 60, 65, 70, 75 or 80 years of age or older. The subject may have suffered from a neurodegenerative disease. The subject may or may not have been treated for the neurodegenerative disease. The subject may be predisposed to a neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease. The Parkinson-plus disease may be selected from the group consisting of multiple system atrophy (MSA), progressive supranuclear palsy (PSP), corticobasal degeneration (CBD), and other Lewy body diseases.

The pharmaceutical composition may further comprise an agent. The agent may be effective for treating or preventing the neurodegenerative disease. Examples of the agent may include L-Dopa/carbidopa (Sinemet), dopamine agonist, MAO-B inhibitors, COMT inhibitors, Amantadine, Anticholinergics, Donepezil, Galantamine, Memantine, Rivastigmine, Namzaric and Adenosine A2a antagonists. The agent may provide a synergistic effect with the specific SENP-1 inhibitor on treatment or prevention of the neurodegenerative disease in the subject. The agent may be effective for changing cells or biological molecules, for example, (a) increasing neurons or SUMO-1, (b) reducing SENP-1, Serine 129 phosphorylated alpha-synuclein or protein aggregates, or (c) reducing SENP-1 nuclear translocalization from cytosol of cells in a region of the brain of a subject.

The pharmaceutical composition may be formulated for oral, sublingual, intranasal, intraocular, rectal, transdermal, mucosal, topical or parenteral administration. For example, the pharmaceutical composition may be administered to the subject orally.

The pharmaceutical compositions may be administered in a variety of unit dosage forms depending upon the method of administration. Suitable unit dosage forms include powders, tablets, pills, capsules, lozenges, suppositories, patches, nasal sprays, injectables, implantable sustained-release formulations, and lipid complexes.

The pharmaceutical composition may be administered to the subject in one or multiple doses, for example, daily. Each dose may contain the specific SENP-1 inhibitor or the SENP-1 nuclear translocalization blocker at about 0.01-5,000, 0.1-5,000, 0.1-1,000 mg, or 0.1-500 mg per kg of the subject's body weight (mg/kg). The pharmaceutical composition may be administered to the subject for at least about 1, 2, 3, 4, 5, 6, 7, 8, 9, 10, 11 or 12 weeks, about 1, 2, 4, 6, 8, 10, 12, 18, 24 or 30 months, or for about 1-12 or 1-10 weeks.

The pharmaceutical composition may further comprise a pharmaceutically acceptable carrier, diluent or excipient. Carriers, diluents and excipients suitable in the pharmaceutical composition are well known in the art. The pharmaceutically acceptable carrier, diluent or excipient may be selected based on the route of administration of the pharmaceutical composition and/or the physio-chemical characteristics of the SENP-1 inhibitor.

The pharmaceutical composition may be sterilized by conventional, well-known sterilization techniques.

The invention also provides a method. The method comprises administering to a subject a pharmaceutical composition, which may comprise a specific SENP-1 inhibitor, in an amount effective for:
(a) treating a neurodegenerative disease in the subject in need thereof;
(b) preventing a neurodegenerative disease in the subject in need thereof;
(c) increasing neurons in a region of the brain of the subject in need thereof;
(d) increasing small ubiquitin-like modifier 1 (SUMO-1) in a region of the brain of the subject in need thereof;
(e) reducing Sentrin-specific protease 1 (SENP-1) in a region of the brain of the subject in need thereof;
(f) reducing Serine 129 phosphorylated alpha-synuclein in a region of the brain of the subject in need thereof; and/or
(g) reducing protein aggregates in a region of the brain of the subject in need thereof.

The method comprises administering to a subject a pharmaceutical composition, which may comprise a SENP-1 nuclear translocalization blocker in an amount effective for reducing nuclear translocalization of SENP-1 from cytosol of cells in a region of the brain of a subject in need thereof.

The method may further comprise blocking mRNA expression of the SENP-1. The SENP-1 mRNA expression may be blocked by a gene therapy using, for example, SiRNAs of SENP-1.

According to the method of the present invention, the specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. For example, the specific SENP-1 inhibitor is momordin lc. The pharmaceutical composition may comprise the specific SENP-1 inhibitor in an amount ranging from 1 mg to 100 g, from 1 mg to 1 g or from 1 gm to 500 mg.

According to the method of the present invention, the pharmaceutical composition may comprise SENP-1 nuclear translocalization blocker in an amount ranging from 1 mg to 100 g, from 1 mg to 1 g or from 1 gm to 500 mg.

The method may comprise administering the pharmaceutical composition to the subject orally, sublingually, intranasally, intraocularly, rectally, trans-dermally, mucosally, topically or parenterally. For example, the pharmaceutical composition may be administered to the subject orally.

The method may comprise administering the pharmaceutical composition to the subject in one or multiple doses, for example, daily. Each dose may contain the specific SENP-1 inhibitor or the SENP-1 nuclear translocalization blocker at about 0.01-5,000, 0.1-5,000, 0.1-1,000 mg, or 0.1-500 mg per kg of the subject's body weight (mg/kg). The method may comprise administering the pharmaceutical composition to the subject for at least about 1, 2, 3, 4, 5, 6, 7, 8, 9, 10, 11 or 12 weeks, about 1, 2, 4, 6, 8, 10, 12, 18, 24 or 30 months, or for about 1-12, 1-10 or 1-4 weeks.

According to the method of the present invention, the subject may be human. The subject may be a male or female. The subject may be an adult. The subject may be 20, 25, 30, 35, 40, 45, 50, 55, 60, 65, 70, 75 or 80 years of age or older. The subject may have suffered from a neurodegenerative disease. The subject may or may not have been treated for the neurodegenerative disease. The subject may be predisposed to a neurodegenerative disease.

According to the method of the present invention, the neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease. The Parkinson-plus disease may be selected from the group consisting of multiple system atrophy (MSA), progressive supranuclear palsy (PSP), corticobasal degeneration (CBD), and other Lewy body diseases.

According to the method of the present invention, the region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex.

The method may be for treating a neurodegenerative disease in a subject in need thereof. The treatment method may comprise administering to the subject an effective amount of a pharmaceutical composition comprising a specific SENP-1 inhibitor. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. For example, the specific SENP-1 inhibitor is momordin lc. The pharmaceutical composition may comprise the specific SENP-1 inhibitor in an amount ranging from 1 mg to 100 g, from 1 mg to 1 g or from 1 mg to 500 mg. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease. The Parkinson-plus disease may be selected from the group consisting of multiple system atrophy (MSA), progressive supranuclear palsy (PSP), corticobasal degeneration (CBD), and other Lewy body diseases. The subject may be a human. The subject may be 20, 25, 30, 35, 40, 45, 50, 55, 60, 65, 70, 75 or 80 years of age or older. The subject may have suffered from a neurodegenerative disease. The subject may or may not have been treated for the neurodegenerative disease.

The treatment method may further comprise mitigating or reversing severity of a symptom associated with the neurodegenerative disease. The symptom may be selected from the group consisting of memory loss, forgetfulness, cognitive decline, apathy, anxiety, agitation, smell loss (anosmia), hallucination, sleep disruption, voice changes, depression, and mood changes.

The treatment method may further comprise improving mobility of the subject. The mobility may be selected from the group consisting of bradykinesia (e.g., limb stiffness), tremor, dyskinesia (e.g., uncontrollable movements), postural instability, nesting, grooming, hindlimb clasping, pole-test, turn time, climb-down time, cross-beam, nesting and rotarod.

The treatment method may further comprise increasing neurons in a region of the brain of the subject. The neuron may be dopaminergic neurons. The dopaminergic neurons may be positive for Tyrosine hydroxylases (TH+). The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex.

The treatment method may further comprise increasing small ubiquitin-like modifier 1 (SUMO-1) in a region of the brain of the subject. The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex.

The treatment method may further comprise reducing Sentrin-specific protease 1 (SENP-1) in a region of the brain of the subject. The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex.

The treatment method may further comprise reducing Serine 129 phosphorylated alpha-synuclein in a region of the brain of the subject. The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex.

The treatment method may further comprise reducing protein aggregates in a region of the brain of the subject. The protein aggregates may be detected by thioflavin-T. The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex.

The method of the present invention may be for preventing a neurodegenerative disease in a subject in need thereof. The prevention method may comprise administering to the subject an effective amount of a pharmaceutical composition comprising a specific SENP-1 inhibitor. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. For example, the specific SENP-1 inhibitor is momordin lc. The pharmaceutical composition may comprise the specific SENP-1 inhibitor in an amount ranging from 1 mg to 100 g, from 1 mg to 1 g or from 1 gm to 500 mg. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease. The Parkinson-plus disease may be selected from the group consisting of multiple system atrophy (MSA), progressive supranuclear palsy (PSP), corticobasal degeneration (CBD), and other Lewy body diseases. The subject may be a human. The subject may be 20, 25, 30, 35, 40, 45, 50, 55, 60, 65, 70, 75 or 80 years of age or older. The subject may be predisposed to the neurodegenerative disease.

The prevention method may further comprise delaying onset of a symptom associated with the neurodegenerative disease in the subject. The symptom may be selected from the group consisting of memory loss, forgetfulness, cognitive decline, apathy, anxiety, agitation, smell loss (anosmia), hallucination, sleep disruption, voice changes, depression, mood changes, and abnormal bowel movements (constipation/diarrhea). The delay of onset of the symptom associated with the neurodegenerative disease may be determined using a control not receiving the pharmaceutical composition.

The prevention method may further comprise delaying onset of motor dysfunction in the subject. The motor dysfunction may be selected from the group consisting of bradykinesia (e.g., limb stiffness), tremor, dyskinesia (e.g., uncontrollable movements), postural instability, nesting, grooming, hindlimb clasping, pole-test, turn time, climb-down time, cross-beam, nesting and rotarod. The delay of onset of the motor dysfunction may be determined using a control not receiving the pharmaceutical composition.

The prevention method may further comprise delaying onset of loss of neurons in a region of the brain of the subject. The delay of onset of the loss of the neurons may be determined using a control not receiving the pharmaceutical composition. The neuron may be dopaminergic neurons. The dopaminergic neurons may be positive for Tyrosine hydroxylases (TH+). The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex.

The prevention method may further comprise delaying onset of decrease of small ubiquitin-like modifier 1 (SUMO-1) in a region of the brain of the subject. The delay of onset of the decrease of the SUMO-1 may be determined using a control not receiving the pharmaceutical composition. The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex.

The prevention method may further comprise delaying onset of increase of Sentrin-specific protease 1 (SENP-1) in a region of the brain of the subject. The delay of onset of the increase of the SENP-1 may be determined using a control not receiving the pharmaceutical composition. The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex.

The prevention method may further comprise delaying onset of increase of Serine 129 phosphorylated alpha-synuclein in a region of the brain of the subject. The delay of onset of the increase of Serine 129 phosphorylated alpha-synuclein may be determined using a control not receiving the pharmaceutical composition. The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex.

The prevention method may further comprise delaying onset of increase protein aggregates in a region of the brain of the subject. The protein aggregates may be detected by Thioflavin-T. The delay of onset of the increase of the protein aggregates may be determined using a control not receiving the pharmaceutical composition. The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex.

The method of the present invention may be for increasing neurons in a region of the brain of a subject in need thereof. The neuron increase method may comprise administering to the subject an effective amount of a pharmaceutical composition comprising a specific SENP-1 inhibitor. The neuron may be dopaminergic neurons. The dopaminergic neurons may be positive for Tyrosine hydroxylases (TH+). The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. For example, the specific SENP-1 inhibitor is momordin lc. The pharmaceutical composition may comprise the specific SENP-1 inhibitor in an amount ranging from 1 mg to 100 g, from 1 mg to 1 g or from 1 mg to 500 mg. The subject may be a human. The subject may be 20, 25, 30, 35, 40, 45, 50, 55, 60, 65, 70, 75 or 80 years of age or older. The subject may have suffered from a neurodegenerative disease. The subject may or may not have been treated for the neurodegenerative disease. The subject may be predisposed to the neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease. The Parkinson-plus disease may be selected from the group consisting of multiple system atrophy (MSA), progressive supranuclear palsy (PSP), corticobasal degeneration (CBD), and other Lewy body diseases.

The method of the present invention may be for increasing small ubiquitin-like modifier 1 (SUMO-1) in a region of the brain of a subject in need thereof. The SUMO-1 increase method may comprise administering to the subject an effective amount of a pharmaceutical composition comprising a specific SENP-1 inhibitor. The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. For example, the specific SENP-1 inhibitor is momordin lc. The pharmaceutical composition may comprise the specific SENP-1 inhibitor in an amount ranging from 1 mg to 100 g, from 1 mg to 1 g or from 1 mg to 500 mg. The subject may be a human. The subject may be 20, 25, 30, 35, 40, 45, 50, 55, 60, 65, 70, 75 or 80 years of age or older. The subject may have suffered from a neurodegenerative disease. The subject may or may not have been treated for the neurodegenerative disease. The subject may be predisposed to the neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease. The Parkinson-plus disease may be selected from the group consisting of multiple system atrophy (MSA), progressive supranuclear palsy (PSP), corticobasal degeneration (CBD), and other Lewy body diseases.

The method of the present invention may be for reducing Sentrin-specific protease 1 (SENP-1) in a region of the brain of a subject in need thereof. The SENP-1 reduction method may comprise administering to the subject an effective amount of a pharmaceutical composition comprising a specific SENP-1 inhibitor. The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. For example, the specific SENP-1 inhibitor is momordin lc. The pharmaceutical composition may comprise the specific SENP-1 inhibitor in an amount ranging from 1 mg to 100 g, from 1 mg to 1 g or from 1 mg to 500 mg. The subject may be a human. The subject may be 20, 25, 30, 35, 40, 45, 50, 55, 60, 65, 70, 75 or 80 years of age or older. The subject may have suffered from a neurodegenerative disease. The subject may or may not have been treated for the neurodegenerative disease. The subject may be predisposed to the neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease. The Parkinson-plus disease may be selected from the group consisting of multiple system atrophy (MSA), progressive supranuclear palsy (PSP), corticobasal degeneration (CBD), and other Lewy body diseases.

The method of the present invention may be for reducing Serine 129 phosphorylated alpha-synuclein in a region of the brain of a subject in need thereof. The Serine 129 phosphorylated alpha-synuclein reduction method may comprise administering to the subject an effective amount of a pharmaceutical composition comprising a specific SENP-1 inhibitor. The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. For example, the specific SENP-1 inhibitor is momordin lc. The pharmaceutical composition may comprise the specific SENP-1 inhibitor in an amount ranging from 1 mg to 100 g, from 1 mg to 1 g or from 1 mg to 500 mg. The subject may be a human. The subject may be 20, 25, 30, 35, 40, 45, 50, 55, 60, 65, 70, 75 or 80 years of age or older. The subject may have suffered from a neurodegenerative disease. The subject may or may not have been treated for the neurodegenerative disease. The subject may be predisposed to the neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease. The Parkinson-plus disease may be selected from the group consisting of multiple system atrophy (MSA), progressive supranuclear palsy (PSP), corticobasal degeneration (CBD), and other Lewy body diseases.

The method of the present invention may be for reducing protein aggregates in a region of the brain of a subject in need thereof. The protein aggregate reduction method may comprise administering to the subject an effective amount of a pharmaceutical composition comprising a specific SENP-1 inhibitor. The protein aggregates may be detected by thioflavin-T. The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex. The specific SENP-1 inhibitor may be selected from the group consisting of small ubiquitin-like modifier 2 (SUMO-2) aldehyde, momordin lc, streptonigrin, hinokiflavone, siRNAs of SENP-1, and a combination thereof. For example, the specific SENP-1 inhibitor is momordin lc. The pharmaceutical composition may comprise the specific SENP-1 inhibitor in an amount ranging from 1 mg to 100 g, from 1 mg to 1 g or from 1 mg to 500 mg. The subject may be a human. The subject may be 20, 25, 30, 35, 40, 45, 50, 55, 60, 65, 70, 75 or 80 years of age or older. The subject may have suffered from a neurodegenerative disease. The subject may or may not have been treated for the neurodegenerative disease. The subject may be predisposed to the neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease. The Parkinson-plus disease may be selected from the group consisting of multiple system atrophy (MSA), progressive supranuclear palsy (PSP), corticobasal degeneration (CBD), and other Lewy body diseases.

The method of the present invention may be for reducing SENP-1 nuclear translocalization from cytosol of cells in a region of the brain of a subject in need thereof. The SENP-1 nuclear translocalization reduction method comprises administering to the subject an effective amount of a pharmaceutical composition comprising a SENP-1 nuclear translocalization blocker. The region of the brain may comprise midbrain, striatum (STR), cortex or a combination thereof. The region of the brain may comprise substantia nigra compacta (SNc) and/or ventral tagmental area (VTA) in midbrain. The region of the brain may comprise STR, SNc in midbrain, or a combination thereof. The region of the brain may comprise midbrain. The region of the brain may comprise cortex. The pharmaceutical composition may comprise the SENP-1 nuclear translocalization blocker in an amount ranging from 1 mg to 100 g, from 1 mg to 1 g or from 1 mg to 500 mg. The subject may be a human. The subject may be 20, 25, 30, 35, 40, 45, 50, 55, 60, 65, 70, 75 or 80 years of age or older. The subject may have suffered from a neurodegenerative disease. The subject may or may not have been treated for the neurodegenerative disease. The subject may be predisposed to the neurodegenerative disease. The neurodegenerative disease may be selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease. The Parkinson-plus disease may be selected from the group consisting of multiple system atrophy (MSA), progressive supranuclear palsy (PSP), corticobasal degeneration (CBD), and other Lewy body diseases.

### Example 1. In Vitro Assays

Cell Viability Assay. The MTT assay is a colorimetric assay that is based on the conversion of water-soluble MTT (3-(4,5-dimethylthiazol-2-yl)-2,5-diphenyltetrazolium bromide) into formazan crystal by mitochondrial dehydrogenase enzymes, soluble in 100% DMSO. The resulting solution is purple in color, and the color intensity is proportional to the viable cells for each well in a 96-well plate. A variety of SENP-1 inhibitors, such as SUMO-2 aldehyde, Momordin lc, streptonigrin, hinokiflavone, oleanolic acid and siRNA of SENP-1, were treated in N27 cells after PFF (1 μg/ml) exposure for 24 h. After adding 5 mM MTT dye to cells, the color intensity in MTT assays was measured using a spectrophotometer (SpectraMax M5e, Molecular devices, Dover, USA), at 570 nm with a reference wavelength of 630 nm. The cells were incubated with the MTT reagent (ab211091, Abcam) at 37° C. for 3 h, followed by solubilizing the formazan crystals in solution.

Cytotoxicity Assay. The lactate dehydrogenase (LDH) is a cytosolic enzyme that is released into the media during cellular stress and increased levels of cytotoxicity. The cytotoxicity assay was measured using reagents from the Pierce LDH Cytotoxicity Assay Kit (88954, Thermo). For the assay, 50 μL of media from each well after PFF exposure, followed by SENP-1 treatment, was put into wells of a new 96-well plate. The LDH reagents were prepared according to the protocol provided, and 50 μL of the LDH reagent was mixed into each well. The total 100 μL of media containing LDH and its reagent were incubated at 37° C. for 30 min, followed by the application of a stop solution. The mixture was analyzed using a spectrophotometer (SpectraMax M5e, Molecular devices) at 490 nm with a reference wavelength of 680 nm.

ROS Levels and Thioflavin-T Assessment. The reactive oxygen species (ROS) is a released byproduct during oxidative stress. On the other hand, thioflavin-T is an assay used to understand the level of protein aggregation that can be detrimental to cell survival. In order to assess the level of oxidative stress, all the SENP-1 inhibitors treated cells were incubated in media containing 5 μg/mL of the ROS reagent CellROX deep red reagent (C10422, Thermo Fisher Scientific) and 25 μg/mL of thioflavin-T reagent (CAS: 2390-54-7, Acros Organics) at 37° C. for 30 min. The cells were then washed 3 times with warm phosphate-buffered saline (PBS) without Ca²⁺ or Mg²⁺ (137 mM NaCl, 2.7 mM KCl, 4.3 mM Na₂HPO₄, and 1.47 mM KH₂PO₄), and several images from each well were collected using EVOS FL Cell Imaging Systems (Invitrogen, MA, USA). A minimum of 2 wells were dedicated to each group, and the experiments were repeated 3-4 times independently (total replicated: n=8-10/group). The intensity of red fluorescence corresponding to ROS levels and green labels corresponding to protein aggregation was analyzed by blinded raters using ImageJ.

### Example 2. Screening Assays

In this study, numerous commercially available SENP-1 inhibitors were tested for the recovery effects from preformed fibrils of alpha-synuclein (PFF)-induced PD models in vitro. The screening process includes several SENP-1 inhibitors, including specific SENP-1 inhibitors (e.g., SUMO-2 Aldehyde, Momordin lc, streptonigrin, hinokiflavonem, and siRNAs of SENP-1) and nonspecific SENP-1 inhibitors (e.g., oleanolic acid, gallic acid), in N27 dopaminergic cells after the exposure of PFF for 24 hrs. Using well-established methods, such as cytotoxicity (LDH) or cell viability (MTT) assay, the measurement of Reactive Oxygen Species (ROS) and protein aggregation in Thioflavin-T staining, we found that several SENP-1 inhibitors including SUMO-2 Aldehyde, Momordin lc, streptonigrin and hinokiflavone clearly showed that those SENP-1 inhibitors are efficacious not only in preventing protein aggregation-mediated damage, but also in reversing PFF-induced damage to nearly healthy or functional state of dopaminergic cells (FIGS. 5-8). However, oleanolic acid and gallic acid did not show an efficacy in all the assays mentioned above.

### Example 3. Neuroprotective Effects of SENP-1 Treatment

The administration of SENP-1 was initially found to protect against in vitro cell death in MPP+ treated N27 dopaminergic cell line and further confirmed against PFF toxicity. In vivo oral Momordin administration (10 or 50 mg/kg) over 6-7 weeks to old mice (15-16 month-old), 3-4 months after PFF injection was sufficient to prevent the accumulation of phosphorylated form of alpha-synuclein in multiple brain regions previously shown to occur as a consequence of PFF injection in mice. The PFF-induced damage has been shown in many different brain regions including the striatum (STR), the Substantia Nigra compacta (SNc) and the cortex (FIG. 12). Importantly, the SENP-1 inhibition was found not only to prevent phosphorylated or misfolded alpha-synuclein accumulation in various brain regions (FIGS. 13-16), but also to protect against neuronal cell loss in this model (FIG. 12).

### Example 4. The Mechanism of SENP-1 Toxicity

After establishing stable N27 cell lines that overexpress WT or NLS mutants (M1 or M2) of human SENP-1 gene, 5×10³ N27 cells were plated in a 96 well-plate and cultured overnight. Then, 1 μg/ml PFF was applied to the stable cell lines for inducing toxicity as described in the MTT and LDH assay sections in the Example 1 above. The location and sequence of SENP-1 nuclear localization sequence 1 (NLS1) are described and indicated with the locations of point mutations, in comparison to WT of human SENP-1 sequence in FIGS. 17A-17B.

### A. Methods and Results

A mouse model of Parkinson's disease is C57BL/6 male or female mice (50% each) injected with 5 μg of mouse PFF at 12 months old, followed by a treatment below for 6-7 weeks:
1) without PFF injection, followed by vehicle (control, n=7);
2) PFF injection, followed by vehicle (PFF only: control, n=8);
3) PFF injection, followed by daily 10 mg/kg Momordin gavage (n=7-8);
4) PFF injection, followed by daily 50 mg/kg Momordin gavage (n=7-8).

Behavioral tests were performed using the methods described in Ghosh et al. (Int J Mol Sci. 2022 Apr. 12; 23 (8): 4262) (FIG. 11A).

Hindlimb Clasping. This is often characterized by bat-like posture when mice are picked up by the tail, indicating they are experiencing severe motor deficits. A mouse was placed on a horizontal plane and held at 2 cm from the tail tip, then slowly to the height of 10 cm from the surface. A video was recorded for 15 sec for each trial. A total of 3 trials were taken over 3 consecutive days and analyzed for the time in hindlimb clasping by a blinded rater.

Rotarod. Rotarod is a behavioral test used to measure motor functionality. For three consecutive days, all the mice were habituated on the rotating rod comprising a horizontal plane rotating at a speed of 4 rotations per minute (rpm), increasing by 2 rpm every minute for 10 min or until they fell off the rod at 20 rpm. The data were collected over three consecutive days where the animals were placed on the rod at 4 rpm, and the rod increased the speed by 1 rpm every 16 sec for 10 min or until the mouse fell off. A longer latency to fall indicates an improved motor functionality.

Pole Test. All the mice were placed on a vertical pole with their face up and habituated to face down the pole to climb down for 5 consecutive days. The time taken for a mouse to turn and position for the descend was noted, and the latency for climb-down was counted. A mouse falling off or slipping down the pole without turning was considered a failure. Videos of the pole test were taken and analyzed by a blinded rater to record the total climb-down or turn time. Minimal 5 trials were performed for each animal, and the average of 5 trials was considered as the final score for data presentation.

Nesting. A 5×5 cm cotton nesting patch was placed in a clean cage housing a single animal. On the following day, pictures were taken of the condition of the nesting material utilized and analyzed in a blind manner. The ability of an animal to tear up the cotton nesting material and build a nest dictates the activity and motor coordination levels of the animal. Three dimensionally built nest was given as 5 points as the highest score, and a barely touched nesting patch was considered a low score of 1 point. The points were represented on the graph to show any improvement in nesting capabilities across the groups.

Grooming. Individual pictures of each animal were taken over 3 days and analyzed for grooming capabilities. Additional data were collected over 6 weeks during the gavaging period for PFF-injected mice to monitor whether the mice in all groups performed grooming activities uniformly. The points were averaged at the end of the assessment after the mice were euthanized. The coat conditions of each animal were assessed to correlate motivation and activity levels for each animal. Dull and non-shiny coats detected showed severe motor deficits, whereas healthy animals spent more time in grooming. The grooming pictures were also analyzed by blinded raters and given a point out of 10, with 1 being a poor/dull coat and 10 being a shiny and healthy-looking coat.

Immunohistochemistry. The serial coronal sections of the right hemisphere were collected throughout the striatum and substantia nigra. Each section with a thickness of 14 μm was mounted as a set of 5 onto positively charged slides (Midwest Sci, Valley Park, MO). The slides were kept at −80° C. until immunohistochemistry was performed. The sections were thawed at room temperature for 30 min, followed by a rehydration step in 0.1M PB for 10 min. The permeabilization step was performed using pre-chilled 100% acetone for 10 min. The excess acetone was dried, and permeabilization solution (40 UL Triton-X in 2% BSA) was added to the slides and kept aside for an additional 10 min. A blocking reagent containing 5% BSA in PB-T was given in the blocking step after drying the slides of excess permeabilization solution. The primary antibody was exposed under the coverslips to prevent the dehydration of the primary antibody. After the primary antibody (EMD Millipore-AB152) treatment at 4° C. overnight, the tissues were washed in PBS-T for 5 min three times. Then, slides were treated with a secondary antibody of Alexa-647-conjugated goat anti-rabbit (1:1000; Molecular Probes, Thermo Fisher Scientific) and incubated at room temperature for 2 h. The secondary antibody was washed in PBS-T for 3×5 min. Coverslips were mounted on slides using ProLong Diamond antifade mounting medium (Thermofisher, MA, USA). All images were captured using EVOS FL Cell Imaging Systems (Invitrogen), and every fifth section was analyzed for measuring the intensity of TH staining in the striatum and counting TH+ neurons in the SNc in a blinded manner. The same procedure was used for assessing the levels of phosphorylated Ser-129-α-synuclein (1:500, Cell signaling, cat #23706S) and protein aggregation using thioflavin-T (20 μM, Acros). Pictures for specific regions such as the striatum and ventral midbrain (vMB) were taken using confocal microscopy and stitched together to obtain a single image for representation purposes. The intensities were measured using ImageJ by a blinded rater.

Immunoblot Analyses. A total of three protein extractions were performed. In the first extraction, a gentle RIPA buffer containing the low concentration of detergent (25 mM Tris-HCL, 150 mM NaCl, 1% NP-40, 1% sodium deoxycholate and 0.1% SDS) was added with phosphatase inhibitors (100 μL for 10 mL) and 5 mM EDTA. The mixture was sonicated and centrifuged at 14,000 rpm for 10 min. The supernatant was collected for measuring the levels of phosphorylated α-syn, SENP-1 or SUMO-1 conjugated proteins for different groups. The pellet was re-dissolved for the second extraction in the RIPA buffer containing 2% SDS, compared with 0.1% SDS, to further solubilize the insoluble phosphorylated α-syn proteins. It was then centrifuged for the third time, and the resulting supernatant was used to load on precast polyacrylamide gel (SurePAGE Bis-Tris, 8-15%, 12 wells; GenScript) and transferred to PVDF membrane (Immobilon-P, EMD Millipore) using the Bio-Rad transfer apparatus. The membranes were further used for estimating the protein levels for each group. The primary antibodies pertaining to each protein analyzed were diluted at 1:1000 (p-Ser-129-α-synuclein, Cell signaling; SENP-1, SAB4501345, EMD) and incubated at 4° C. overnight. On the following day, the membranes were washed and treated with the secondary antibody (1:2000, Invitrogen-31462) at room temperature for 2 h, prior to developing the blots in the Immobilon Forte Western HRP substrate (WBLUF0500, EMD Millipore) for the detection under the ChemiDoc iBright CL1000 (Invitrogen 1000). Equal loading was estimated by stripping the membrane and re-probing against β-actin (1:4000; Invitrogen MA5-15739-HRP) and then incubated with secondary (1:8000, Invitrogen-31432) for 2 h. Molecular weights for each protein were checked using the PageRuler pre-stained protein ladder on the immunoblots (EMP Millipore-MPSTD4; Thermofisher-26616). ImageJ was used to analyze specific bands on each blot and measured against the loading control (β-actin) for normalizing each band intensity.

### B. Neuroprotective Effects of SENP-1 Treatment

The administration of SENP-1 was initially found to protect against in vitro cell death in MPP+ treated N27 dopaminergic cell line and further confirmed against PFF toxicity. In vivo oral Momordin administration (10 or 50 mg/kg) over 6-7 weeks to old mice (15-16 month-old), 3-4 months after PFF injection was sufficient to prevent the accumulation of phosphorylated form of alpha-synuclein in multiple brain regions previously shown to occur as a consequence of PFF injection in mice. The PFF-induced damage has been shown in many different brain regions including the striatum (STR), the Substantia Nigra compacta (SNc) and the cortex (FIG. 12). Importantly, the SENP-1 inhibition was found not only to prevent phosphorylated or misfolded alpha-synuclein accumulation in various brain regions (FIGS. 13-16), but also to protect against neuronal cell loss in this model (FIG. 12).

Immunohistochemistry. The serial coronal sections of the right hemisphere were collected throughout the striatum and substantia nigra. Each section with a thickness of 14 μm was mounted as a set of 5 onto positively charged slides (Midwest Sci, Valley Park, MO). The slides were kept at −80° C. until immunohistochemistry was performed. The sections were thawed at room temperature for 30 min, followed by a rehydration step in 0.1M PB for 10 min. The permeabilization step was performed using pre-chilled 100% acetone for 10 min. The excess acetone was dried, and permeabilization solution (40 μL Triton-X in 2% BSA) was added to the slides and kept aside for an additional 10 min. A blocking reagent containing 5% BSA in PB-T was given in the blocking step after drying the slides of excess permeabilization solution. The primary antibody was exposed under the coverslips to prevent the dehydration of the primary antibody. After the primary antibody (EMD Millipore-AB152) treatment at 4° C. overnight, the tissues were washed in PBS-T for 5 min three times. Then, slides were treated with a secondary antibody of Alexa-647-conjugated goat anti-rabbit (1:1000; Molecular Probes, Thermo Fisher Scientific) and incubated at room temperature for 2 h. The secondary antibody was washed in PBS-T for 3×5 min. Coverslips were mounted on slides using ProLong Diamond antifade mounting medium (Thermofisher, MA, USA). All images were captured using EVOS FL Cell Imaging Systems (Invitrogen), and every fifth section was analyzed for measuring the intensity of TH staining in the striatum and counting TH+ neurons in the SNc in a blinded manner. The same procedure was used for assessing the levels of phosphorylated Ser-129-α-synuclein (1:500, Cell signaling, cat #23706S) and protein aggregation using thioflavin-T (20 μM, Acros). Pictures for specific regions such as the striatum and ventral midbrain (vMB) were taken using confocal microscopy and stitched together to obtain a single image for representation purposes. The intensities were measured using ImageJ by a blinded rater.

Immunoblot Analyses. A total of three protein extractions were performed. In the first extraction, a gentle RIPA buffer containing the low concentration of detergent (25 mM Tris-HCL, 150 mM NaCl, 1% NP-40, 1% sodium deoxycholate and 0.1% SDS) was added with phosphatase inhibitors (100 μL for 10 mL) and 5 mM EDTA. The mixture was sonicated and centrifuged at 14,000 rpm for 10 min. The supernatant was collected for measuring the levels of phosphorylated α-syn, SENP-1 or SUMO-1 conjugated proteins for different groups. The pellet was re-dissolved for the second extraction in the RIPA buffer containing 2% SDS, compared with 0.1% SDS, to further solubilize the insoluble phosphorylated α-syn proteins. It was then centrifuged for the third time, and the resulting supernatant was used to load on precast polyacrylamide gel (SurePAGE Bis-Tris, 8-15%, 12 wells; GenScript) and transferred to PVDF membrane (Immobilon-P, EMD Millipore) using the Bio-Rad transfer apparatus. The membranes were further used for estimating the protein levels for each group. The primary antibodies pertaining to each protein analyzed were diluted at 1:1000 (p-Ser-129-α-synuclein, Cell signaling; SENP-1, SAB4501345, EMD) and incubated at 4° C. overnight. On the following day, the membranes were washed and treated with the secondary antibody (1:2000, Invitrogen-31462) at room temperature for 2 h, prior to developing the blots in the Immobilon Forte Western HRP substrate (WBLUF0500, EMD Millipore) for the detection under the ChemiDoc iBright CL1000 (Invitrogen 1000). Equal loading was estimated by stripping the membrane and re-probing against β-actin (1:4000; Invitrogen MA5-15739-HRP) and then incubated with secondary (1:8000, Invitrogen-31432) for 2 h. Molecular weights for each protein were checked using the PageRuler pre-stained protein ladder on the immunoblots (EMP Millipore-MPSTD4; Thermofisher-26616). ImageJ was used to analyze specific bands on each blot and measured against the loading control (β-actin) for normalizing each band intensity.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A pharmaceutical composition comprising a specific Sentrin-specific protease 1 (SENP-1) inhibitor in an amount effective for:
(a) treating a neurodegenerative disease in a subject;
(b) preventing a neurodegenerative disease in a subject;
(c) increasing neurons in a region of the brain of a subject;
(d) increasing small ubiquitin-like modifier 1 (SUMO-1) in a region of the brain of a subject;
(e) reducing Sentrin-specific protease 1 (SENP-1) in a region of the brain of a subject;
(f) reducing Serine 129 phosphorylated alpha-synuclein in a region of the brain of a subject; and/or
(g) reducing protein aggregates in a region of the brain of a subject.

2. The pharmaceutical composition of claim 1, wherein the region of the brain comprises midbrain, striatum (STR), cortex or a combination thereof.

3. The pharmaceutical composition of claim 1, wherein the subject is a human.

4. The pharmaceutical composition of claim 1, wherein the subject is predisposed to or has suffered from a neurodegenerative disease.

5. (canceled)

6. The pharmaceutical composition of claim 1, wherein the neurodegenerative disease is selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease.

7. The pharmaceutical composition of claim 1, wherein the specific SENP-1 inhibitor is selected from the group consisting of small ubiquitin-like modifier 2 (SUMO-2) aldehyde, momordin lc, streptonigrin, hinokiflavone, siRNAs of SENP-1, and a combination thereof.

8. The pharmaceutical composition of claim 1, wherein the composition comprises the specific SENP-1 inhibitor in an amount ranging from 1 mg to 100 g.

9. A method for treating a neurodegenerative disease in a subject in need thereof, comprising administering to the subject an effective amount of a pharmaceutical composition comprising a specific Sentrin-specific protease 1 (SENP-1) inhibitor.

10. The method of claim 9, wherein the neurodegenerative disease is selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease.

11. The method of claim 9, further comprising at least one of:
mitigating severity of a symptom associated with the neurodegenerative disease;
reversing the severity of the symptom;
improving mobility of the subject;
increasing neurons in a region of the brain of the subject;
increasing small ubiquitin-like modifier 1 (SUMO-1) in a region of the brain of the subject;
reducing Sentrin-specific protease 1 (SENP-1) in a region of the brain of the subject;
reducing Serine 129 phosphorylated alpha-synuclein in a region of the brain of the subject; and/or
reducing protein aggregates in a region of the brain of the subject.

12. (canceled)

13. (canceled)

14. (canceled)

15. The method of claim 11, wherein at least one of:
the region of the brain comprises midbrain, striatum (STR), cortex, or a combination thereof;
the subject is a human; or
the subject has suffered from the neurodegenerative disease.

16. (canceled)

17. (canceled)

18. A method for preventing a neurodegenerative disease in a subject in need thereof, comprising administering to the subject an effective amount of a pharmaceutical composition comprising a specific Sentrin-specific protease 1 (SENP-1) inhibitor.

19. The method of claim 18, wherein the neurodegenerative disease is selected from the group consisting of Parkinson's disease, Alzheimer's disease, Amyotrophic Lateral Sclerosis, Huntington's disease, Spinal muscular atrophy, Friedreich ataxia, Dementia with Lewy body and Parkinson-plus disease.

20. The method of claim 18, further comprising at least one of:
delaying onset of a symptom associated with the neurodegenerative disease in the subject;
delaying onset of motor dysfunction in the subject;
delaying onset of loss of neuron in a region of the brain of the subject;
delaying onset of decrease of small ubiquitin-like modifier 1 (SUMO-1) in a region of the brain of the subject;
delaying onset of increase of Sentrin-specific protease 1 (SENP-1) in a region of the brain of the subject;
delaying onset of increase of serine 129 phosphorylated alpha-synuclein in a region of the brain of the subject; or
delaying onset of increase of protein aggregates in a region of the brain of the subject.

21. (canceled)

22. (canceled)

23. The method of claim 20, wherein at least one of: the region of the brain comprises midbrain, striatum (STR), cortex, or a combination thereof;
the region of the brain comprises striatum (STR), substantia nigra compacta (SNc) in midbrain, or a combination thereof the subject is a human; or
the subject is predisposed to the neurodegenerative disease.

24. (canceled)

25. (canceled)

26. The method of claim 9, wherein the subject is in need of increasing neurons in a region of the brain of a subject in need thereof, comprising administering to the subject an effective amount of a pharmaceutical composition comprising a specific Sentrin-specific protease 1 (SENP-1) inhibitor.

27. The method of claim 9, wherein the subject is in need of increasing small ubiquitin-like modifier 1 (SUMO-1) in a region of the brain of a subject in need thereof, comprising administering to the subject an effective amount of a pharmaceutical composition comprising a specific Sentrin-specific protease 1 (SENP-1) inhibitor.

28. The method of claim 9, wherein the subject is in need of reducing Sentrin-specific protease 1 (SENP-1) in a region of the brain of a subject in need thereof, comprising administering to the subject an effective amount of a pharmaceutical composition comprising a specific Sentrin-specific protease 1 (SENP-1) inhibitor.

29. The method of claim 9, wherein the subject is in need of reducing Serine 129 phosphorylated alpha-synuclein in a region of the brain of a subject in need thereof, comprising administering to the subject an effective amount of a pharmaceutical composition comprising a specific Sentrin-specific protease 1 (SENP-1) inhibitor.

30. The method of claim 9, wherein the subject is in need of reducing protein aggregates in a region of the brain of a subject in need thereof, comprising administering to the subject an effective amount of a pharmaceutical composition comprising a specific Sentrin-specific protease 1 (SENP-1) inhibitor.

31. The method of claim 9, wherein the subject is in need of reducing nuclear translocalization of Sentrin-specific protease 1 (SENP-1) from cytosol of cells in a region of the brain of a subject in need thereof, comprising administering to the subject an effective amount of a pharmaceutical composition comprising a SENP-1 nuclear translocalization blocker.

32. (canceled)

33. (canceled)

34. (canceled)

35. (canceled)

36. (canceled)

37. (canceled)

38. (canceled)

39. (canceled)

40. The method of claim 9, wherein the specific SENP-1 inhibitor is selected from the group consisting of small ubiquitin-like modifier 2 (SUMO-2) aldehyde, momordin lc, streptonigrin, hinokiflavone, and a combination thereof.

41. The method of claim 9, further comprising blocking mRNA expression of the SENP-1.

42. The method of claim 9, further comprising administering the pharmaceutical composition to the subject orally.

43. The method of claim 9, further comprising administering the pharmaceutical composition to the subject at a dose of the specific SENP-1 inhibitor at 1-500 mg per kg of the subject's body weight.

44. The method of claim 9, further comprising administering the pharmaceutical composition to the subject at a dose of the specific SENP-1 inhibitor at 1-500 mg per kg of the subject's body weight daily for 1-10 weeks.
